(19) United States
(12) Patent Application Publication
(10) Pub. No.: US 2016/0222122 A1
(43) Pub. Date: Aug. 4, 2016

(54) INTEGRIN-MODULATING THERAPIES FOR TREATING FIBROTIC DISEASE

(71) Applicant: The Johns Hopkins University, Baltimore, MD (US)

(72) Inventors: Harry C. Dietz, Towson, MD (US); Elizabeth E. Gerber, Baltimore, MD (US)

(73) Assignee: The Johns Hopkins University, Baltimore, MD (US)

(21) Appl. No.: 15/022,098

(22) PCT Filed: Sep. 15, 2014
(86) PCT No.: PCT/US14/55631

§ 371 Date: Mar. 15, 2016

Related U.S. Application Data
(60) Provisional application No. 61/878,000, filed on Sep. 15, 2013.

(51) Int. Cl. C07K 16/28

(52) U.S. Cl. CPC C07K 16/2842; C07K 16/2848; A61K 2039/505

(57) ABSTRACT

A method of treating scleroderma and other fibrotic conditions is disclosed. The method includes the step of administering to a subject having a fibrotic condition an effective amount of an agent capable of modulating the activity of one or more integrins. The integrin activity-modulating agent alters cell-matrix interactions, thus reducing the symptoms of the fibrotic condition.

### CROSS-REFERENCE TO RELATED APPLICATIONS

This applications claims the benefit of U.S. provisional Application No. 61/878,000 filed on Sep. 15, 2013, which is incorporated by reference herein.

### STATEMENT REGARDING FEDERALLY SPONSORED RESEARCH OR DEVELOPMENT

This invention was made with government support under RO1-AR41135 and PO1-AR049698 awarded by the National Institutes of Health. The government has certain rights in the invention.

### FIELD OF THE INVENTION

The invention relates generally to methods of treating scleroderma and other fibrotic diseases using therapies that modulate the activity of integrins, thereby altering cellular-matrix interactions.

### BACKGROUND

Scleroderma is a poorly understood disease characterized by pathological fibrosis and hardening of the skin. In systemic sclerosis (SSc), a common and etiologically mysterious form of scleroderma, previously healthy adults acquire fibrosis of the skin and viscera in association with the production of autoantibodies. SSc affects about 1 in 5,000 individuals in the United States. Familial recurrence is extremely rare, and causal genes have not been identified. While the onset of fibrosis in SSc typically correlates with the production of autoantibodies, whether they contribute to disease pathogenesis or simply serve as a marker of disease remains controversial, and the mechanism for antibody induction is largely unknown. Other types of scleroderma include stiff skin syndrome (SSS), an autosomal dominant congenital form of scleroderma caused by a mutation in a specific domain of the gene encoding fibrillin 1.

Similar fibrotic conditions may occur in organs other than the skin. For example, idiopathic pulmonary fibrosis (IPF), which does not respond well to any known medical therapy, is characterized by chronic fibrosis and associated inflammation of the lungs.

There is a need in the art for new therapies that can effectively treat these and other fibrotic diseases.

### SUMMARY

The inventors have determined that cell-matrix interactions can be an important therapeutic target for fibrosis and related fibrotic conditions. Specifically, agents that affect and/or interact with one or more integrins can be used to effectively treat the symptoms of fibrosis.

Accordingly, in a first aspect, the disclosure encompasses a method of treating a fibrotic disease or condition. The method includes the step of administering to a subject having a fibrotic disease or condition an effective amount of an integrin activity-modulating agent. Such treatment reduces the symptoms of the fibrotic disease or condition.

In some embodiments, the integrin activity-modulating agent includes manganese. In some embodiments, the integrin activity-modulating agent includes an integrin-activating agent or an integrin-blocking agent.

In some embodiments, the integrin activity-modulating agent includes an antibody or a peptide that is capable of interacting with one or more integrins.

In some such embodiments, the antibody or peptide is an integrin-activating antibody or peptide, including without limitation a β1 integrin-activating antibody or peptide, such as a β1 integrin-activating antibody (β1aAb). Non-limiting examples of a β1aAb that could be used include 9EG7 and TS2/16.

In other such embodiments, the antibody or peptide is an integrin-blocking antibody or peptide, including without limitation a β3 integrin-blocking antibody or peptide, such as a β3 integrin-blocking antibody (β3bAb).

Non-limiting examples of fibrotic diseases or conditions for which the method could be used include scleroderma, including without limitation stiff skin syndrome and systemic sclerosis, and idiopathic pulmonary fibrosis.

In a second aspect, the disclosure encompasses an integrin activity-modulating agent for use in treating a fibrotic disease or condition. In some embodiments, the integrin activity-modulating agent includes manganese. In some embodiments, the integrin activity-modulating agent includes an integrin-activating agent or an integrin-blocking agent.

In some embodiments, the integrin activity-modulating agent includes an antibody or a peptide that is capable of interacting with one or more integrins.

In some such embodiments, the antibody or peptide is an integrin-activating antibody or peptide. A non-limiting example is a β1 integrin-activating antibody or peptide, such as a β1 integrin-activating antibody (β1aAb). Non-limiting examples of β1aAb include 9EG7 and TS2/16.

In other such embodiments, the antibody or peptide is an integrin-blocking antibody or peptide. A non-limiting example is a β3 integrin-blocking antibody or peptide, such as a β3 integrin-blocking antibody (β3bAb).

Non-limiting examples of fibrotic diseases or conditions that could be treated with the integrin activity-modulating agent include scleroderma, including without limitation stiff skin syndrome and systemic sclerosis, and idiopathic pulmonary fibrosis.

In a third aspect, the disclosure encompasses an integrin activity-modulating agent for use in manufacturing a medicament for treating a fibrotic disease or condition. In some embodiments, the integrin activity-modulating agent includes manganese. In some embodiments, the integrin activity-modulating agent includes an integrin-activating agent or an integrin-blocking agent.

In some embodiments, the integrin activity-modulating agent includes an antibody or a peptide that is capable of interacting with one or more integrins.

In some such embodiments, the antibody or peptide is an integrin-activating antibody or peptide. A non-limiting example is a β1 integrin-activating antibody or peptide, such as a β1 integrin-activating antibody (β1aAb). Non-limiting examples of β1aAb include 9EG7 and TS2/16.

In other such embodiments, the antibody or peptide is an integrin-blocking antibody or peptide. A non-limiting example is a β3 integrin-blocking antibody or peptide, such as a β3 integrin-blocking antibody (β3bAb).

Non-limiting examples of fibrotic diseases or conditions that could be treated with the medicament include scleroderma, including without limitation stiff skin syndrome and systemic sclerosis, and idiopathic pulmonary fibrosis.

Other objects, features and advantages of the present invention will become apparent after review of the specification, claims and drawings.

### BRIEF DESCRIPTION OF THE FIGURES

FIG. 1. SSS mouse models show skin fibrosis. Masson's trichrome staining of back skin sections from male mice (genotypes indicated) at 1 month (top left panels) and 3 months (bottom left panels) of age demonstrates progressive loss of subcutaneous fat and an expanded zone of dense dermal collagen in mutant animals. Quantification of the thickness of the zones of dermal collagen and subcutaneous fat in wild-type and mutant mice at 1 (top right panels) and 3 (bottom right panels) months of age is shown. Similar findings were observed in mutant female mice (FIGS. 6A and 6B). 1 month males: n=9 (+/+), 10 (WC/+), 10 (WC/WC), 9 (DE/+); 3 month males: n=13 (+/+), 9 (WC/+), 9 (WC/WC), 9 (DE/+). Scale bars, 50 μm. * p<0.05, ** p<0.01, † p<0.001, ‡ p<0.0001. DE=D1545E. WC=W1572C.

FIG. 2. Integrin-modulating interventions prevent skin fibrosis. (A) Flow cytometry of cells derived from the dermis reveals a unique population expressing both α5β1 and active β3 integrins (monitored using WOW-1 antibody) in mutant mice that is eliminated upon treatment with β1aAb but not an isotypematched control (IgG). Representative contour plots are shown. An agonist and antagonist of β3 integrin activation were used to attest to the specificity of the WOW-1 antibody (FIG. 20B). Isotype control-treated: n=5 (Fbn1^{+/+}), 7 (Fbn1^{D1545E/+}); β1aAb-treated: n=4 (Fbn1+), 7 (Fbn1^{D1545E/+}). (B) Clinical assessment demonstrated that β1aAb prevented skin stiffness in mutant animals when compared to those treated with an isotype-matched control (IgG). (C) Masson's trichrome staining reveals reduced skin collagen and preservation of subcutaneous fat in β1aAb-treated mutants. Isotype control-treated: n=12 (Fbn1^{+/+}), 9 (Fbn1^{D1545E/+}), 8 (Fbn1^{W1572C/+}); β1aAb-treated: n=12 (Fbn1^{+/+}), 10 (Fbn1^{D1545E/+}), 10 (Fbn1^{W1572C/+}). DE=D1545E. WC=W1572C.

FIG. 3. A panspecific transforming growth factor β-neutralizing antibody reverses established skin fibrosis. (A) Clinical assessment showing that stiffness was fully normalized by TGFβ-neutralizing antibody (TGFβNAb) treatment, commencing at three months of age and lasting twelve weeks. (B) Histologic and morphometric analyses using Masson's trichrome stain. Isotype control-treated: n=14 (Fbn1^{+/+}), 9 (Fbn1^{D1545E/+}), 8 (Fbn1^{W1572C/+}). TGFβNAb treated: n=14 (Fbn1^{+/+}); 10 (Fbn1^{D1545E/+}); 8 (Fbn1^{W1572C/+}). DE=D1545E. WC=W1572C. Scale bars, 50 μm. * p<0.05, ** p<0.01, † p<0.001, ‡ p<0.0001.

FIG. 4. Immunologic abnormalities in SSS mice are prevented by integrin-modulating therapies. (A) Increased circulating levels of anti-nuclear and anti-topoisomerase I antibodies by enzyme-linked immunosorbent assay (ELISA) in Fbn1^{D1545E/+} mice at 3 months of age are normalized upon treatment with β1aAb but not an isotype-matched control (IgG). Isotype control-treated: n=6 (Fbn1^{+/+}), 4 (Fbn1^{D1545E/+}); β1aAb-treated: n=4 (Fbn1^{+/+}), 10 (Fbn1^{D1545E/+}). (B) The cells expressing high α5β1 integrin in the dermis of mutant mice are CD317(high) cells that fail to accumulate upon treatment with β1aAb but not an isotype-matched control (IgG). The CD317(high) cells that accumulate in the dermis of mutant mice are B220(+)CD3(−)CD19(−) plasmacytoid dendritic cells and (C) express both IFNα and IL-6. For panels B-C: Isotype control-treated: n=5 (Fbn1^{+/+}), 7 (Fbn1^{D1545E/+}); β1aAb-treated: n=4 (Fbn1^{+/+}), 7 (Fbn1^{D1545E/+}). For other panel: n=5 (Fbn1^{+/+}), 4 (Fbn1^{D1545E/+}), 4 (Fbn1^{DW1572C/+}). DE=D1545E. WC=W1572C. * p<0.05, ** p<0.01, † p<0.001, ‡ p<0.0001.

FIG. 5. (A) Schematic of constructs used to generate Fbn1^{D1545E/+} and Fbn1^{W1572C/+} mice by homologous recombination. The construct contained a neomycin resistance cassette (NeoR), flanked by loxP sites, that was later removed via breeding to mice expressing Cre-recombinase. (B) Representative Southern blot (for mutation W1572C) showing proper targeting in embryonic stem (ES) cells prior to blastocyst injection and implantation into pseudopregnant mice. (C) Mice were genotyped on the basis of creation of a new AciI site (W1572C) or destruction of a BsmAI site (D1545E) in correctly targeted mice. Fbn1 genotypes: WC/+, Fbn1^{DW1572C/+}; DE/+, (Fbn1^{D1545E/+}).

FIG. 6. (A) Masson's trichrome staining of back skin sections from mutant (genotypes indicated) female mice at 1 month (top panels) and 3 months (bottom panels) of age demonstrates progressive loss of subcutaneous fat and an expanded zone of dense dermal collagen. (B) Quantification of the thickness of the zones of dermal collagen and subcutaneous fat in wild-type and mutant female mice at 1 (top panels) and 3 (bottom panels) months of age. 1 month females: n=8 (+/+), 8 (WC/+), 8 (WC/WC), 9 (DE/+); 3 month females: n=12 (+/+), 10 (WC/+), 9 (WC/WC), 9 (DE/+). Scale bars, 50 μm. * p<0.05, ** p<0.01, † p<0.001, ‡ p<0.0001. (C) Electron microscopy shows excessive microfibrillar deposits (black arrows) and sparsely distributed electron-dense (black) elastin in mutant skin. Scale bars, 500 nm.

FIG. 7. Flow cytometry analysis showing that cell-surface expression of total αvβ3 or αvβ5 were normal in SSS mice and did not change with β1aAb treatment.

FIG. 8. (A) Schematic showing how the stretched skin area/total surface area (SSA/TSA) ratios were measured. Mice were anesthetized and their back-hair removed. Mice were then briefly suspended by their back skin and photographed in profile in a uniform manner. (B) Mutant mice showed a reduced SSA/TSA ratio that was normalized upon treatment with β1aAb but not by an isotype-matched control (IgG). (C) There were no differences in body weight between all experimental groups. Isotype control-treated: n=12 (Fbn1^{+/+}), 9 (Fbn1^{D1545E/+}), 8 (Fbn1^{W1572C/+}); β1aAb-treated: n=12 (Fbn1^{+/+}), 10 (Fbn1^{D1545E/+}), 10 (Fbn1^{W1572C/+}). Measurements were performed with NIH image J software (National Institute of Health, Bethesda, Md., USA). * p<0.05, ** p<0.01, † p<0.001, ‡ p<0.0001.

FIG. 9. (A) Introduction of haploinsufficiency (−/+) or the null state (−/−) for the gene encoding integrin β3 (Itgb3) attenuates or prevents skin stiffening, respectively, in mouse models of SSS. (B) Histologic and morphometric analyses using Masson's trichrome stain. (C) A small subset of mice (˜12% overall) that are haploinsufficient (−/+) or null (−/−) for Itgb3, the gene encoding integrin β3, show focal epidermal hyperplasia and increased cellularity and collagen in the dermis at five months of age. These findings were observed irrespective of Fbn1 genotype. Scale bars, 50 μg. n=12 (Fbn1^{+/+} and Itgb3^{+/+}), 13 (Fbn1^{+/+} and Itgb3^{-/+}), 7 (Fbn1^{+/+} and Itgb3^{-/-}), 8 (Fbn1^{D1545E/+} and Itgb3^{+/+}), 18 (Fbn1^{D1545E/+} and Itgb3^{-/+}), 14 (Fbn1^{D1545E/+} and Itgb3^{-/-}), 7 (Fbn1^{W1572C/+} and Itgb3^{+/+}), 9 (Fbn1^{W1572C/+} and Itgb3^{-/+}), 6 (Fbn1^{W1572C/+} and Itgb3^{-/-}). Scale bars, 50 μc. * p<0.05, ** p<0.01, † p<0.001, ‡ p<0.0001.

FIG. 10. (A) Flow cytometry analysis did not reveal an increase in the expression of integrins known to potently support the activation of TGFβ (αvβ5, αvβ6 or αvβ8) in the dermis of mutant mice, when compared to wild-type littermates. (B) Immunofluorescence analysis reveals increased latency associated peptide (LAP)-1, LAP-2 and total TGFβ2 in the dermis of mutant mice, when compared to wild-type littermates. No difference in active (free) TGFβ1 was observed. n=5 (Fbn1^{+/+}, +/+), 4 (Fbn1^{D1545E/+}, DE/+), 4 (Fbn1^{DW1572C+}, WC/+). Scale bars, 50 μm.

FIG. 11. Increased circulating levels of anti-nuclear and anti-topoisomerase I antibodies by enzyme-linked immunosorbent assay (ELISA) in mutant mice at 18 months of age. n=5 (Fbn1^{+/+}, +/+), 4 (Fbn1^{D1545E/+}, DE/+), 4 (Fbn1^{DW1572/+}, WC/+). * p<0.05, ** p<0.01, † p<0.001, ‡ p<0.0001.

FIG. 12. (A) Increased deep dermal expression of active integrin β3 in the mutant skin colocalizes with CD45(+) cells derived from the bone marrow; both signals were normalized upon treatment with β1aAb but not with an isotypematched control (IgG). Isotype control-treated: n=5 (Fbn1^{+/+}), 7 (Fbn1^{D1545E/+}); β1aAb-treated: n=4 (Fbn1^{+/+}), 7 (Fbn1^{D1545E/+}). For panel E: n=5 (Fbn1^{+/+}), 4 (Fbn1^{D1545E/+}), 4 (Fbn1^{DW1572C/+}). * p<0.05, ** p<0.01, † p<0.001, ‡ p<0.0001. (B) Gating strategy for pDC identification: First, live cells are gated. Next, CD11b(+) leukocytes, CD3(+) T cells, and CD19(+) B cells are excluded. (C) Representative flow cytometry plots showing that the cells under consideration are B220+, CD317(high), Siglec H(+), Ly6C(high) and show a conventional size distribution for pDCs. (D) Immunofluorescent staining confirming the presence of Siglec H(+) cells in the dermis of placebo-treated SSS mice, but absent in that of of wild-type or treated animals. Scale bars, 50 μm. Isotype control-treated: n=5 (Fbn1^{+/+}), 7 (Fbn1^{D1545E/+}); β1aAb-treated: n=4 (Fbn1^{+/+}), 7 (Fbn1^{D1545E/+}). For other panel: n=5 (Fbn1^{+/+}), 4 (Fbn1^{D1545E/+}), 4 (Fbn1^{DW1572C/+}). * p<0.05, ** p<0.01, † p<0.001, ‡ p<0.0001. (E) CD11b(−)CD3(−)CD19(−)CD317(high) cells are expressing interferon α, as expected for activated pDCs, as well as interleukin-6. Percentages shown indicate % of total dermal cells. n=5 (Fbn1^{+/+}, +/+), 4 (Fbn1^{D1545E/+}, DE/+), 4 (Fbn1^{DW1572C/+} WC/+). * p<0.05, ** p<0.01, † p<0.001, ‡ p<0.0001.

FIG. 13. (A) The skewing of T helper (Th) CD4(+) lymphocytes toward IL-4(+) Th2 and IL-17(+) Th17 populations in mutant mice was prevented upon treatment with β1aAb. Isotype control-treated: n=5 (Fbn1^{+/+}), 7 (Fbn1^{D1545E/+}); β1aAb treated: n=4 (Fbn1^{+/+}), 7 (Fbn1^{D1545E/+}). For other panel: n=5 (Fbn1^{+/+}), 4 (Fbn1^{D1545E/+}), 4 (Fbn1^{DW1572C/+}). * p<0.05, ** p<0.01, † p<0.001, ‡ p<0.0001. (B) Representative flow cytometry plots of dermal cells positive for CD3 and interleukins 17, 9, 22, 4, and 13. (C) Quantification by boxplot shows increases in CD3(+) cells also positive for interleukins 17, 9, 22, 4, and 13, and in CD3(−) cells also positive for interleukins-9 or interleukin-22. (D) There were no changes in either FoxP3(+) CD4(+)T-regulatory (Treg) or IFNγ(+) CD4(+)Th1 cells in the dermis of SSS mice. All mice were male and 2 months of age. n=5 (Fbn1^{+/+}, +/+), 4 (Fbn1^{D1545E/+}, DE/+), 4 (Fbn1^{DW1572C/+}, WC/+). * p<0.05, ** p<0.01, † p<0.001, ‡ p<0.0001.

FIG. 14. Mutant mice showed accumulation of B220(high)CD19(+) activated B cells and CD138(+)B220(low)CD19(+) plasma cells in the dermis that was prevented by treatment with β1aAb but not an isotype-matched control (IgG). Isotype control-treated: n=5 (Fbn1^{+/+}), 7 (Fbn1^{D1545E/+}); β1aAb-treated: n=4 (Fbn1^{+/+}), 7 (Fbn1^{D1545E/+}). For other panel: n=5 (Fbn1^{+/+}), 4 (Fbn1^{D1545E/+}), 4 (Fbn1^{DW1572C/+}). * p<0.05, ** p<0.01, † p<0.001, ‡ p<0.0001.

FIG. 15. (A) TGF-β neutralizing antibody (TGFβNAb) reverses accumulation of pDCs (defined by B220(+)CD3(−)CD19(−)) in the dermis of Fbn1^{D1545E/+} mice, and (B) the expression of both IFNα and IL-6 in these cells. Both (C) the skewing of T helper (Th) CD4(+) lymphocytes toward IL-4(+) Th2 and IL-17(+) Th17 populations, and (D) the accumulation of B220(high)CD19(+) activated B cells and CD138(+)B220(low)CD19(+) plasma cells in the dermis of Fbn1^{D1545E/+} mice were reversed upon treatment with TGFβNab, but not an isotype-matched control (IgG). Isotype control-treated: n=4 (Fbn1^{+/+}), 4 (Fbn1^{D1545E/+}). TGFβNAb-treated: n=4 (Fbn1^{+/+}); 4 (Fbn1^{D1545E/+}). * p<0.05, ** p<0.01, † p<0.001, ‡ p<0.0001.

FIG. 16. Adherence and activation of plasmacytoid dendritic cells (pDCs) in vitro. (A) Wild-type spleen-derived pDCs show an increase in adherence to the matrix elaborated by murine embryonic fibroblasts (MEFs) derived from Fbn1^{W1572C/+} (WC/+, n=8) and Fbn1^{W1572C/W1572C} (WC/WC, n=4) SSS mice, when compared to Fbn1^{+/+} (+/+, n=6) mice. (B) Among adherent pDCs, those plated on mutant MEFs show increased expression of WOW-1, integrin α5pβ1, IL-6, and IFNα. * p<0.05, ** p<0.01, † p<0.001, ‡ p<0.0001.

FIG. 17. Cultured SSc dermal fibroblasts show increased total β1 integrin by flow cytometry. Treatment with β3 integrin-blocking antibody (β3bAb) did not significantly reduce cell-surface presentation of total β1 integrin. Quantifications reflect the analysis of 6 control and 5 SSc cell lines. * p<0.05.

FIG. 18. Expression and signaling abnormalities in SSc fibroblasts are attenuated by integrin-modulating antibodies. (A) Cultured primary SSc fibroblasts show high surface expression of WOW-1 that was normalized by treatment with β1aAb. Representative flow cytometry histograms depict the percent of maximum (y-axis) at various fluorescent intensities (x-axis). Quantification of the percent of positive cells is also shown. Total αvβ3 and αvβ5 were normal in SSc cells and did not change with treatment. (B) Cultured SSc fibroblasts show low expression of microRNA-29a (miR-29a) and high expression of messenger RNAs (mRNAs) derived from the genes encoding types IA2 (COL1A2) and III (COL3A1) collagens, when compared to age- and gender-matched control fibroblasts (far left bar in each graph); each of these abnormalities was normalized upon treatment with β1aAb in a dose-dependent manner. (C) SD208, an antagonist of the kinase activity of the type I TGFβ receptor subunit (TβRI), normalizes expression of the genes encoding type IA2 and III collagens in primary dermal fibroblasts derived from patients with SSc. Although treatment increased expression of miR-29a, this finding did not reach significance. (D, E) Control fibroblasts show phosphorylation of Smad3 (pSMAD3) in response to 5 minutes of stimulation with TGFβ1, without a change in phosphorylated extracellular regulated kinase1/2 (pERK1/2). Neither signaling cascade was attenuated by β1aAb, β3bAb or β1 integrin-blocking antibody (β1bAb). In contrast, SSc fibroblasts uniquely show ERK1/2 activation (pERK1/2) in response to TGFβ1 that was normalized after treatment with β1aAb or β3bAb but not β1bAb. Both Smad3 and ERK1/2 activation were sensitive to treatment with SD208, an antagonist of the kinase activity of the type I TGFβ receptor subunit (TβRI). (F) U0126, an inhibitor of the mitogen-activated protein kinase/ERK kinase (MEK), increases miR-29a expression and reduces type IA2 and III collagen expression in SSc fibroblasts. Quantifications for panels A-F reflect the analysis of 6 control and 5 SSc cell lines. * p<0.05, ** p<0.01, † p<0.001, ‡ p<0.0001. (G) Flow cytometry reveals that CD317(+) pDCs show high phosphorylation of ERK1/2 (pERK) in SSS mouse models; n=5 (Fbn1^{+/+}), 4 (Fbn1^{D1545E/+}), 4 (Fbn1^{DW1572C/+}). (H,I) MEK inhibitor RDEA119 prevents skin stiffness, dermal collagen accumulation and loss of subcutaneous fat in Fbn1^{D1545E/+} mice. For panels G-I, Isotype control-treated: n=11 (Fbn1^{+/+}), 10 (Fbn1^{D1545E/+}); RDEA119-treated: n=10 (Fbn1^{+/+}), 10 (Fbn1^{D1545E/+}). * p<0.05, ** p<0.01, † p<0.001, ‡ p<0.0001.

FIG. 19. Events influencing and influenced by plasmacytoid dendritic cells (pDCs). The abnormal extracellular matrix (ECM) in SSS leads to concentration of TGFβ in the skin. TGFβ can induce expression of itself and IL-6 by pDCs; the combination of TGFβ and IL-6 leads to Th17 skewing. pDCs also secrete type I interferon (IFN-α/β), which together with IL-6 can induce Th1 polarization and the activation/maturation of plasma cells and autoreactive B cells. IFN-α/β can also induce myeloid dendritic cells (mDCs) to phagocytize cellular debris, which can indirectly contribute to autoantibody production (dashed arrow). pDCs can also contribute to Th2 polarization through secretion of OX40L or IL-4 and the Th2 cytokines IL-4 and IL-13 can influence pDC performance. The expression of integrins by pre-pDCs, perhaps in response to an altered ECM, can influence their transmigration, adhesion and/or maturation to pDCs.

FIG. 20. (A) There were no differences in final blood cell counts between isotype control- and β1aAb-treated animals. n=3 for each experimental group. Nml Range=the normal values reported by the Comparative Pathology Laboratory at Johns Hopkins University School of Medicine. K/μL=thousands per cubic microliter of blood. M/μL=millions per cubic microliter of blood. (B) Specificity of the WOW-1 antibody for integrin αvβ3 in its active conformation was assessed in control fibroblasts by flow cytometry. As expected, chelation of calcium with 10 mM Ethylenediaminetetraacetic acid (EDTA)—an event known to prevent the active conformation of αvβ3—reduced immunoreactivity, while treatment with 2 mM MnCl₂—known to activate αvβ3—increased immunoreactivity.

### DETAILED DESCRIPTION

### I. In General

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. The terms “a” (or “an”), “one or more” and “at least one” can be used interchangeably herein. The terms “comprising,” “including,” and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods and materials of specific embodiments are now described. All publications mentioned herein are incorporated herein by reference for all purposes, including for describing and disclosing the chemicals, cell lines, vectors, animals, instruments, statistical analysis and methodologies which are reported in the publications which might be used in connection with the disclosed methods.

A sequence listing in computer readable form is submitted with this application, and is hereby incorporated by reference herein.

### II. The Invention

Integrins are transmembrane receptor proteins that mediate the attachment between a cell and its surroundings, such as other cells or the extracellulat matrix. They are involved in cell signaling and the regulation of the cell cycle, shape, and motility. Integrins have been extensively studied. There are many different types of integrins, and multiple types may appear on the same cell surface.

Methods of utilizing integrin-modulating agents to effectively treat and/or prevent a fibrotic disease or condition in a subject are disclosed herein. Such integrin-modulating agents may include without limitation antibodies, peptides, and metal ions (such as manganese) that are know in the art to interact with integrins. Integrin-modulating agents may block, inactivate, activate, or otherwise change the integrin's native activity. Non-limiting examples of integrin-modulating agents that can be used in the disclosed method are illustrated in the Example below. Assays to measure the integrin-modulating ability of a given agent are well known to a person skilled in the art.

The disclosed methods include the use of pharmaceutically acceptable salts of the integrin-modulating agents. As used herein, the term “pharmaceutically acceptable salt” refers to a compound formulated from a base compound which achieves substantially the same pharmaceutical effect as the base compound.

The disclosed method may utilize derivatives of known integrin-modulating agents. The term “derivatives” includes but is not limited to ether derivatives, acid derivatives, amide derivatives, ester derivatives and the like. In addition, this method may utilizing hydrates of the integrin-modulating agents. The term “hydrate” includes but is not limited to hemihydrate, monohydrate, dihydrate, trihydrate and the like.

As used herein, the term “treating” includes preventative as well as disorder remittent treatment. As used herein, the terms “reducing,” “suppressing” and “inhibiting” have their commonly understood meaning of lessening or decreasing.

As used herein, the term “administering” refers to bringing any part of a subject, including without the subject's tissue, organs or cells, in contact with the integrin-modulating agent. A “subject” refers to a mammal, preferably a human, that either: (1) has a fibrotic disease or condition remediable or treatable by administering an integrin-modulating agent; or (2) is susceptible to a fibrotic disease or condition that is preventable by administering an integrin-modulating agent.

In one embodiment, the disclosed methods comprise administering an integrin-modulating agent as the sole active ingredient. Also encompassed by the disclosed methods is administering the integrin-modulating agent in combination with one or more other therapeutic agents, or as part of a pharmaceutical composition.

As used herein, “pharmaceutical composition” means a therapeutically effective amounts of the integrin-modulating agent together with suitable diluents, preservatives, solubilizers, emulsifiers, and adjuvants, collectively “pharmaceutically-acceptable carriers.” As used herein, the terms “effective amount” and “therapeutically effective amount” refer to the quantity of active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives.

In this case, an amount would be deemed therapeutically effective if it resulted in one or more of the following: (a) the prevention of a fibrotic disease or condition, (b) the reversal or stabilization of a fibrotic disease or condition, or (c) the reduction of symptoms associated with a fibrotic disease or condition. The optimum effective amount can be readily determined by one of ordinary skill in the art using routine experimentation.

Pharmaceutical compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, marmitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The disclosed methods also include administering particulate compositions coated with polymers (e.g., poloxamers or poloxamines). Other embodiments of the compositions incorporate particulate forms protective coatings, protease inhibitors or permeation enhancers for various routes of administration, including topical, parenteral, pulmonary, nasal and oral. In one embodiment, the pharmaceutical composition is administered parenterally, paracancerally, transmucosally, tansdermally, intramuscularly, intravenously, intradermally, subcutaneously, intraperitonealy, intraventricularly, intracranially and intratumorally.

Further, as used herein “pharmaceutically acceptable carriers” are well known to those skilled in the art and include, but are not limited to, 0.01-0.1M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions administerable according to the disclosed method include formulation in lipophilic depots (e.g. fatty acids, waxes, oils). The methods may also use particulate compositions coated with polymers (e.g. poloxamers or poloxamines) and the compound coupled to antibodies directed against tissue-specific receptors, ligands or antigens or coupled to ligands of tissue-specific receptors.

Optionally, a pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. EngI. J. Med. 321:574 (1989)). In another embodiment, polymeric materials can be used. In yet another embodiment, a controlled release system can be placed in proximity to the therapeutic target, i.e., the prostate, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990).

The pharmaceutical preparation can comprise the integrin-modulating agent alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the agent can be administered to a patient by, for example, subcutaneous implantation of a pellet. In a further embodiment, a pellet provides for controlled release of antiandrogen compound over a period of time. The preparation can also be administered by intravenous, intraarterial, or intramuscular injection of a liquid preparation oral administration of a liquid or solid preparation, or by topical application. Administration can also be accomplished by use of a rectal suppository or a urethral suppository.

The pharmaceutical preparations can be prepared by known dissolving, mixing, granulating, or tablet-forming processes. For oral administration, the anti-androgens or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the anti-androgen compounds or their physiologically tolerated derivatives such as salts, esters, N-oxides, and the like are converted into a solution, suspension, or expulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active agent is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof. In addition, the compositions can contain auxiliary substances such as wetting or emulsifying agents, or pH buffering agents which enhance the effectiveness of the active ingredient.

An active component can be formulated into the composition as neutralized pharmaceutically acceptable salt forms. Pharmaceutically acceptable salts include the acid addition salts, which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the integrin-modulating agent or its physiologically tolerated derivatives such as salts, esters, N-oxides, and the like are prepared and applied as solutions, suspensions, or emulsions in a physiologically acceptable diluents, with or without a pharmaceutical carrier.

In another embodiment, the active agent can be delivered in a vesicle, in particular a liposome (see Langer, Science 249:1527-1533 10 (1990); Treat et al., in Liposomes in the Therapy of Infectious Disease and Cancer, Lopez-Berestein and Fidler (eds.), Liss, N.Y., pp. 353-365 (1989); Lopez-Berestein ibid., pp. 317-327).

For use in medicine, the salts of the integrin-mediating agents may be pharmaceutically acceptable salts. Other salts may, however, be useful in the preparation of the compounds according to the invention or of their pharmaceutically acceptable salts. Suitable pharmaceutically acceptable salts of the compounds include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

The following Example is offered by way of illustration only, and not by way of limitation.

### EXAMPLE

### Integrin Modulating Therapies Prevent Fibrosis and Autoimmunity in Genetic Mouse Models of Scleroderma

Introduction

In systemic sclerosis (SSc), a common and etiologically mysterious form of scleroderma (defined as pathologic fibrosis of the skin), previously healthy adults acquire fibrosis of the skin and viscera in association with autoantibodies. SSc affects about 1 in 5,000 individuals in the United States [1]. Familial recurrence is extremely rare and causal genes have not been identified. While the onset of fibrosis in SSc typically correlates with the production of autoantibodies, whether they contribute to disease pathogenesis or simply serve as a marker of disease remains controversial and the mechanism for their induction is largely unknown [2]. The study of SSc is hindered by a lack of animal models that faithfully recapitulate the etiology of this complex disease.

To gain a foothold in the pathogenesis of pathologic skin fibrosis, we chose to study stiff skin syndrome (SSS), a rare but tractable Mendelian disorder that shows childhood onset of diffuse skin fibrosis with autosomal dominant inheritance and complete penetrance. We showed that SSS is caused by heterozygous missense mutations in the gene encoding fibrillin-1, the major constituent of extracellular microfibrils [3]. Notably, SSS mutations all localize to the only domain in fibrillin-1 that harbors an Arg-Gly-Asp (RGD) motif needed to mediate cell-matrix interactions by binding to cell-surface integrins [3].

General Procedures, Results, and Discussion.

Here we show that mouse lines that harbor analogous amino acid substitutions in fibrillin-1 recapitulate aggressive skin fibrosis that is prevented by integrin-modulating therapies and reversed by antagonism of the pro-fibrotic cytokine transforming growth factor β (TGFβ). Mutant mice show skin infiltration of pro-inflammatory immune cells including plasmacytoid dendritic, T helper, and plasma cells, and autoantibody production; these findings are normalized by integrin-modulating therapies or TGFβ antagonism. These data show that alterations in cell-matrix interactions are sufficient to initiate and sustain inflammatory and pro-fibrotic programs and highlight novel therapeutic strategies.

Fibrillin-1 contributes to the regulation of TGFβ, a cytokine that has been descriptively linked to many fibrotic diseases including both SSS and SSc [3, 4]. TGFβ is secreted from the cell in the context of a large latent complex (LLC) that includes the active cytokine bound to a dimer of its processed N-terminal propeptide, latency-associated peptide (LAP), which in turn binds to latent TGFβ-binding proteins (LTBPs) [5]. Studies in mouse models and in vitro have shown that fibrillin-1 directly interacts with LTBPs, allowing sequestration of the LLC by microfibrils [5].

Mutations throughout the FBN1 gene also cause Marfan Syndrome (MFS), a disorder characterized by bone overgrowth, ocular lens dislocation, and aortic dilatation [6]. Failed matrix sequestration of the LLC in fibrillin-1-deficient MFS patients and mice promotes increased activation of and signaling by TGFβ. SSS mutations are specifically localized to the 4th transforming growth factor-β binding protein-like domain (TB4) of fibrillin-1, which encodes the RGD motif through which fibrillin-1 binds integrins αvβ3, α5β1, and αvβ6 [3, 5].

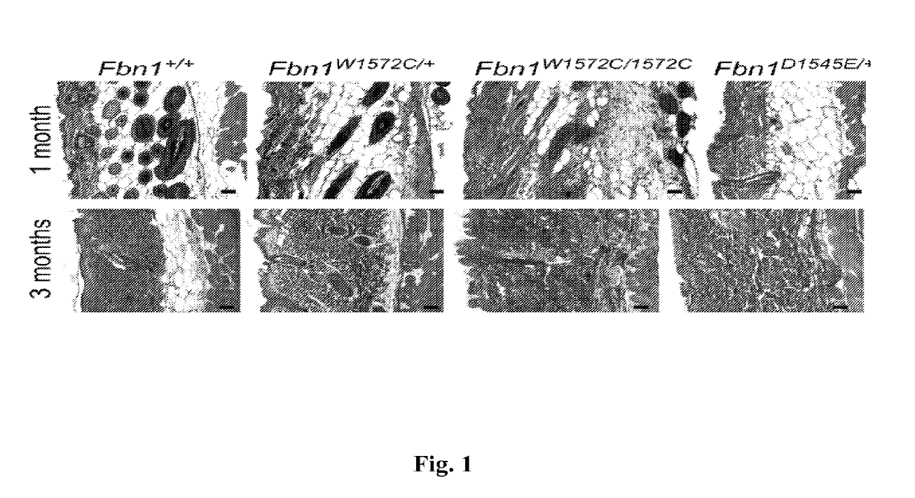

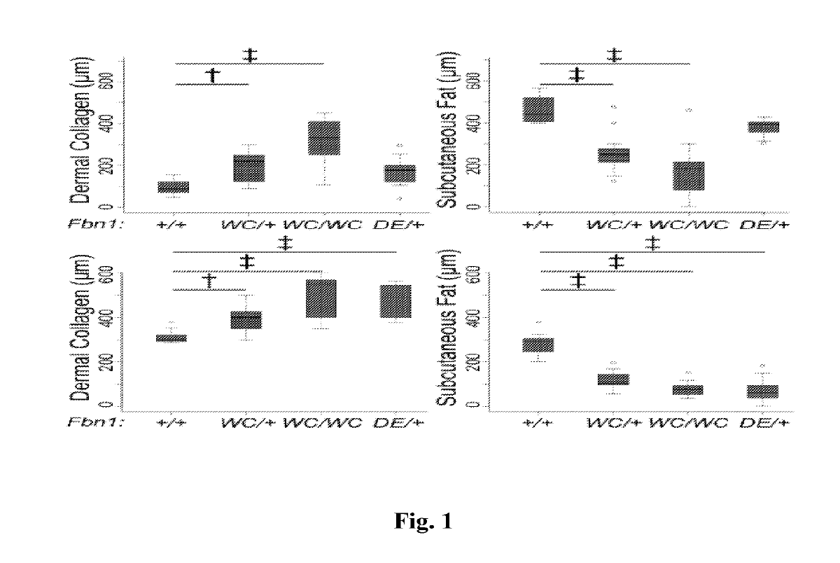

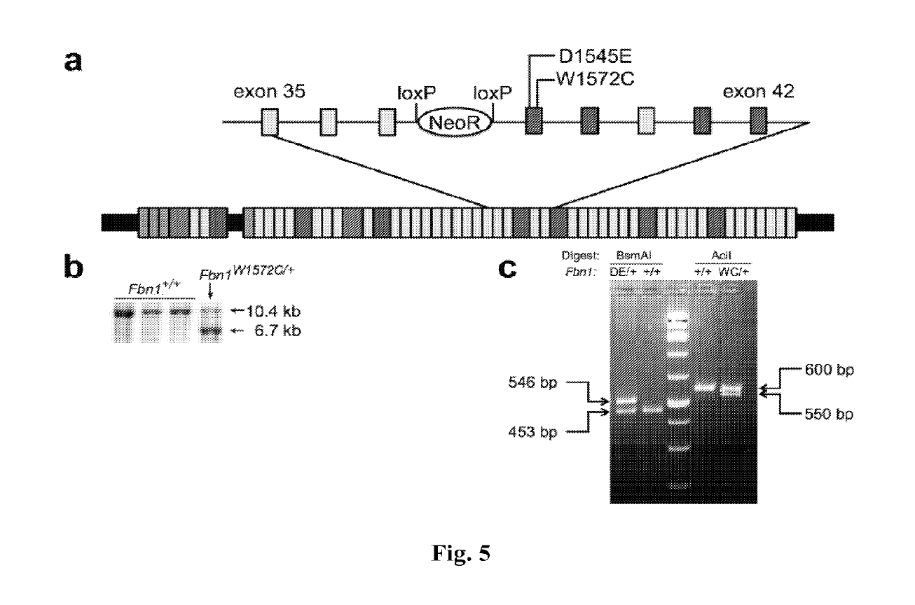

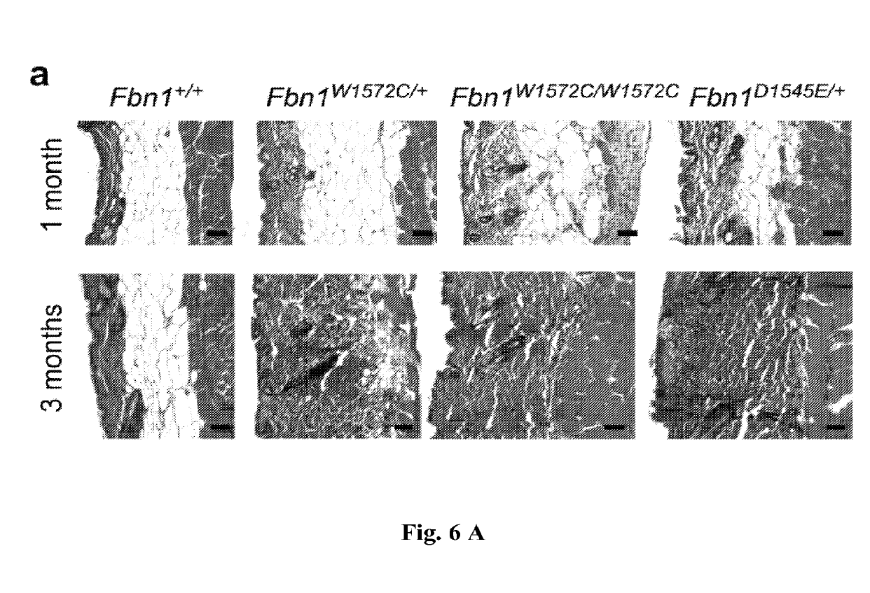

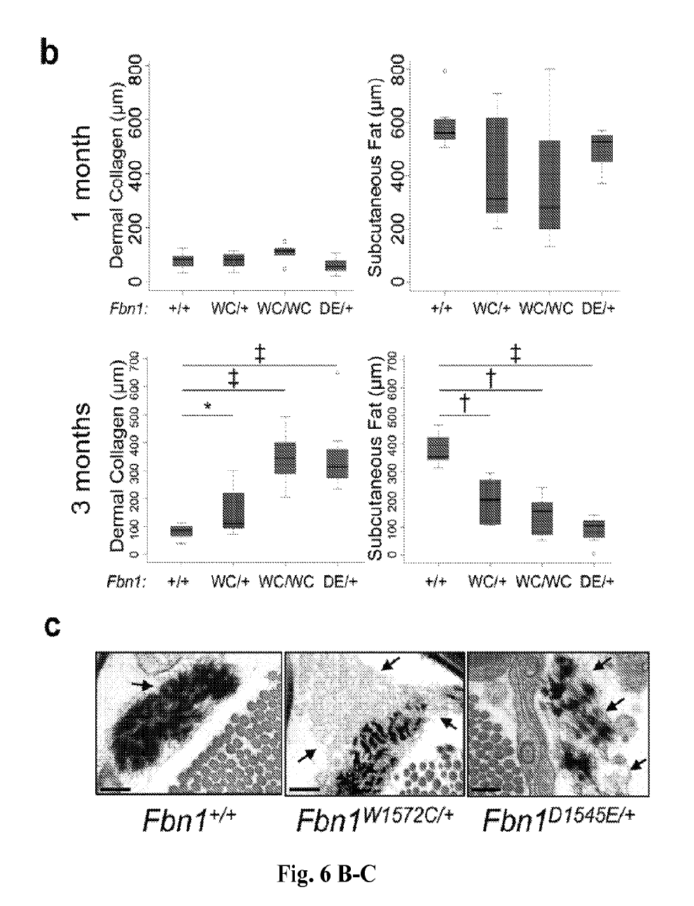

To determine if failed interaction between integrins and fibrillin-1 is sufficient to initiate skin fibrosis, two Fbn1-targeted knock-in mouse models were generated: one with SSS-associated change W1572C (the mouse equivalent of human W1570C) and the other with an RGD to RGE substitution (D1545E) predicted to cause an obligate loss of integrin binding to fibrillin-1 (FIG. 5). Mice heterozygous for either mutation phenocopy SSS with increased deposition of collagen by 1 month of age and dramatic reduction of subcutaneous fat by three months of age (FIGS. 1, 6A, and 6B). While homozygosity for D1545E causes embryonic lethality before embryonic day 10.5, mice homozygous for W1572C are viable and show accelerated skin fibrosis when compared to heterozygous littermates (FIGS. 1, 6A, and 6B).

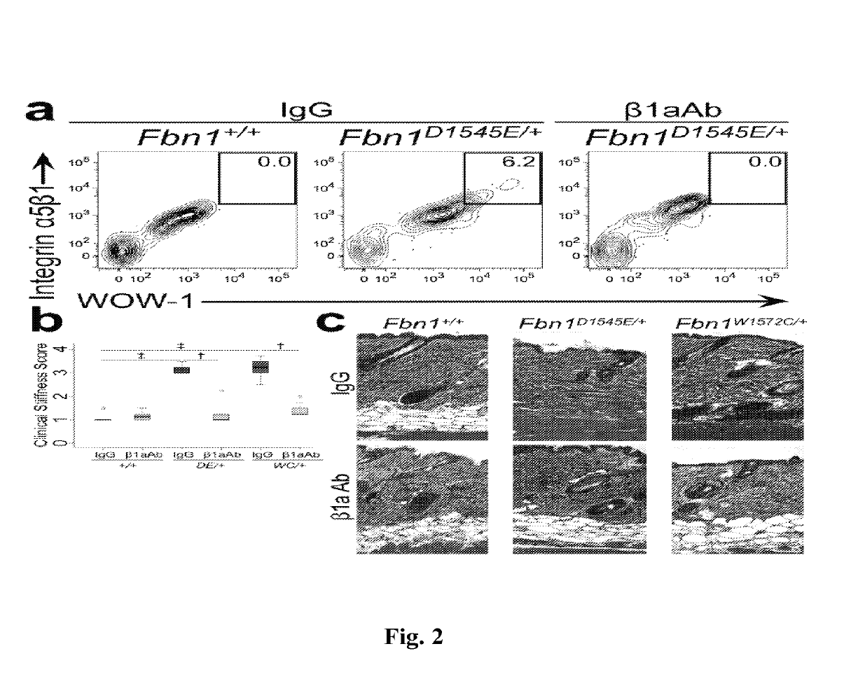

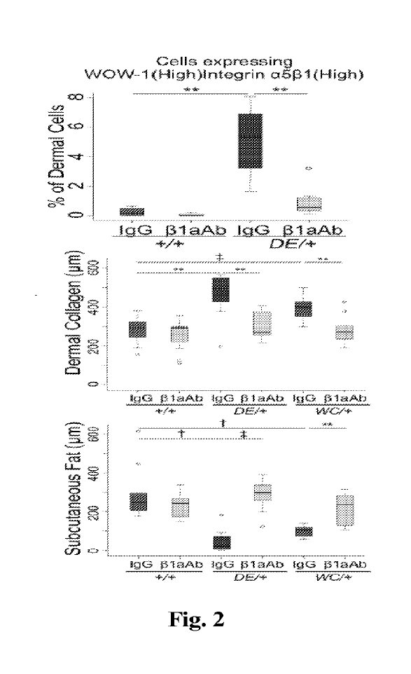

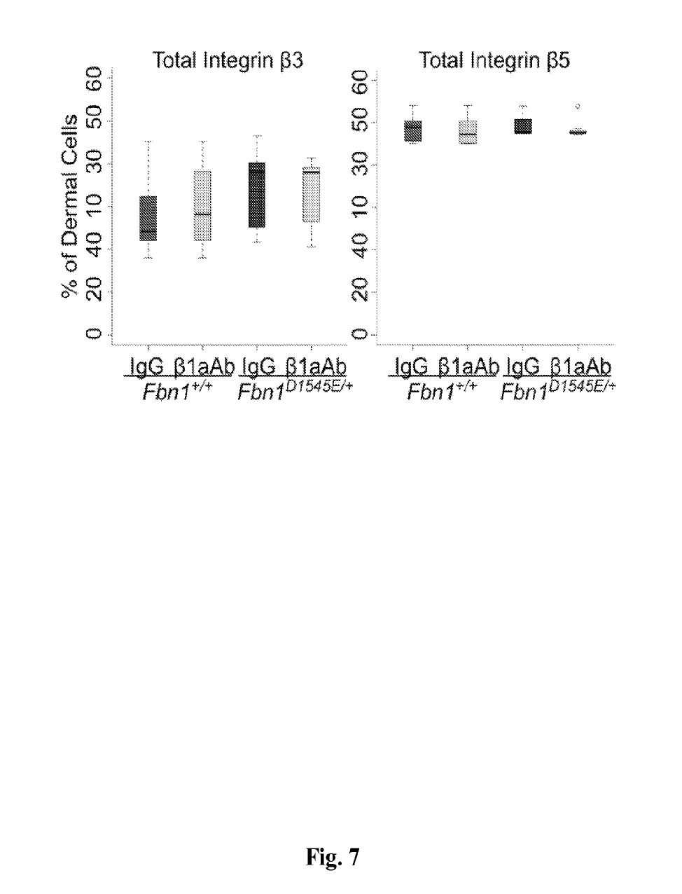

As seen in patients with SSS or SSc [3], mutant mice show disorganized and excessive microfibrillar aggregates in the dermis with sparsely distributed elastin (FIG. 6C). Freshly isolated cells from mutant dermis show increased surface levels of integrin α5β1 and integrin αvβ3 in its active conformation (as assessed using WOW-1 antibody) by flow cytometry (FIG. 2A). There was no corresponding increase in either total β3 integrin or integrin β5, a subtype that can cross-react with WOW-1 (FIG. 7). Based on these data, we hypothesized that disrupted cell-matrix interaction in SSS results in compensatory upregulation of specific integrins at the surface of dermal cells, and that integrins represent a possible therapeutic target for this disease.

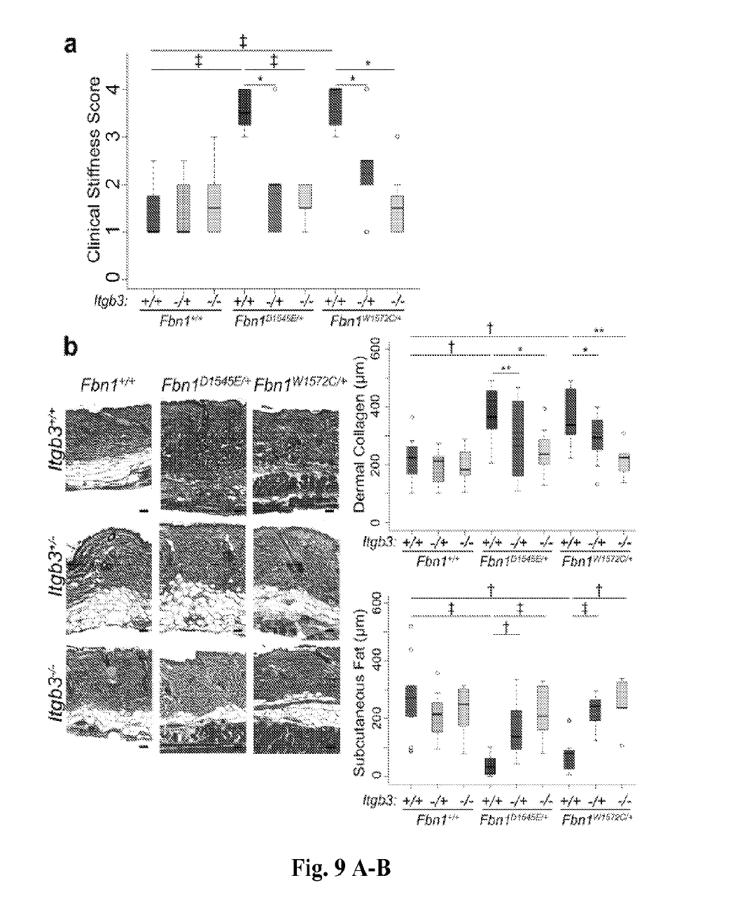

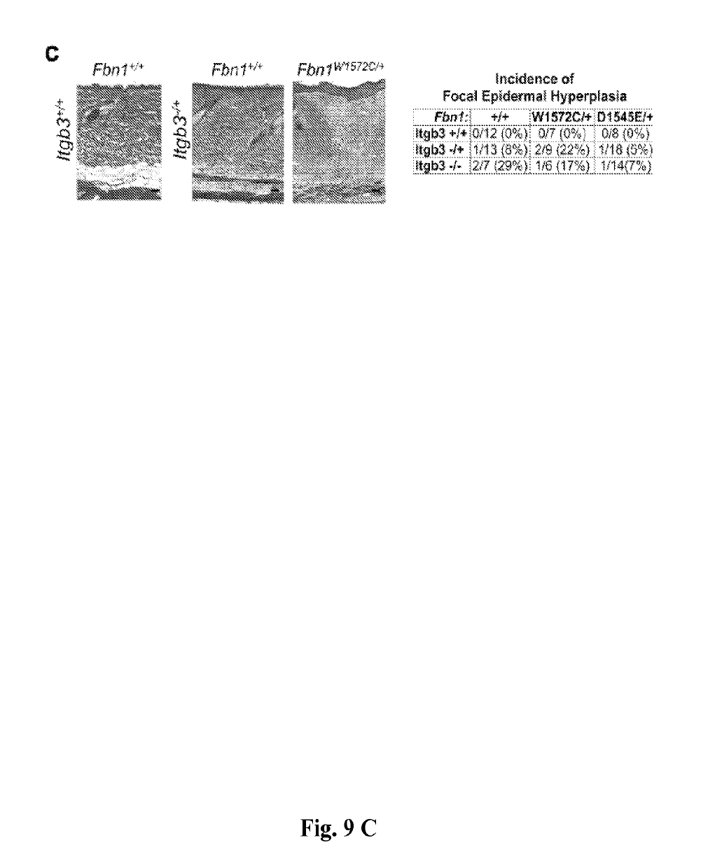

We next investigated whether mimicking integrin-matrix ligand (i.e. fibrillin-1) interactions in mutant mice using a β1 integrin-activating antibody (β1aAb, 9EG7) offered therapeutic potential for the treatment of SSS. Twelve weeks of β1aAb treatment normalized integrin expression, skin stiffness and distensibility and skin architecture in SSS mouse models (FIGS. 2A-C and 8). To determine the relevance of reduced expression of active β3 integrin to this phenotypic rescue, we tested whether targeted introduction of haploinsufficiency or complete deficiency for β3 integrin could diminish or prevent skin fibrosis. Strikingly, SSS mice deficient for β3 integrin showed normalized skin stiffness, collagen deposition and subcutaneous fat by three months of age (FIGS. 9A and 9B). By five months of age, 8 of 67 (12%) of Itgb3-targeted animals developed focal dermal and epidermal thickening (irrespective of Fbn1 genotype) reminiscent of the aberrant wound healing previously described in β3 integrin-deficient mice (FIG. 9C) [7].

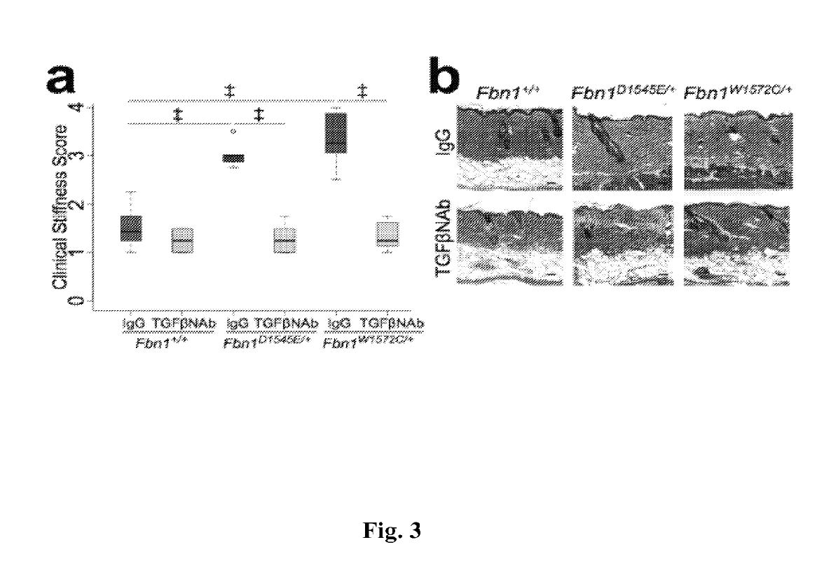

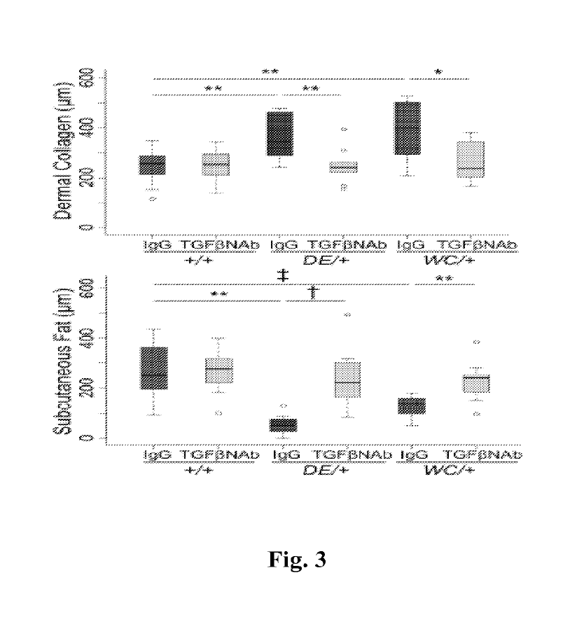

To assess for a pathogenic contribution for TGFβ, SSS mice were treated for twelve weeks with a panspecific TGFβ neutralizing antibody (NAb) or isotypematched control IgG after establishment of dense fibrosis at twelve weeks of age. Clinical (FIG. 3A) and histological (FIG. 3B) findings confirmed full reversal of skin stiffness and restoration of skin architecture in NAb-treated animals. Potential mechanisms for enhanced TGFβ activity include excessive concentration of latent TGFβ by the abnormally abundant microfibrillar aggregates in the dermis, or excessive integrin-mediated activation (release) of TGFβ from its latent complex [8].

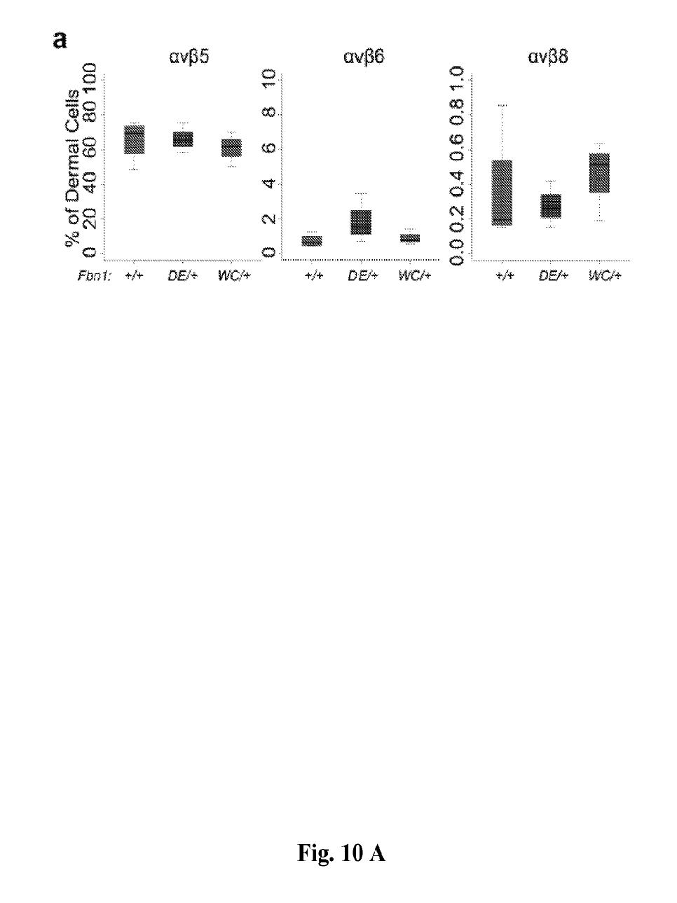

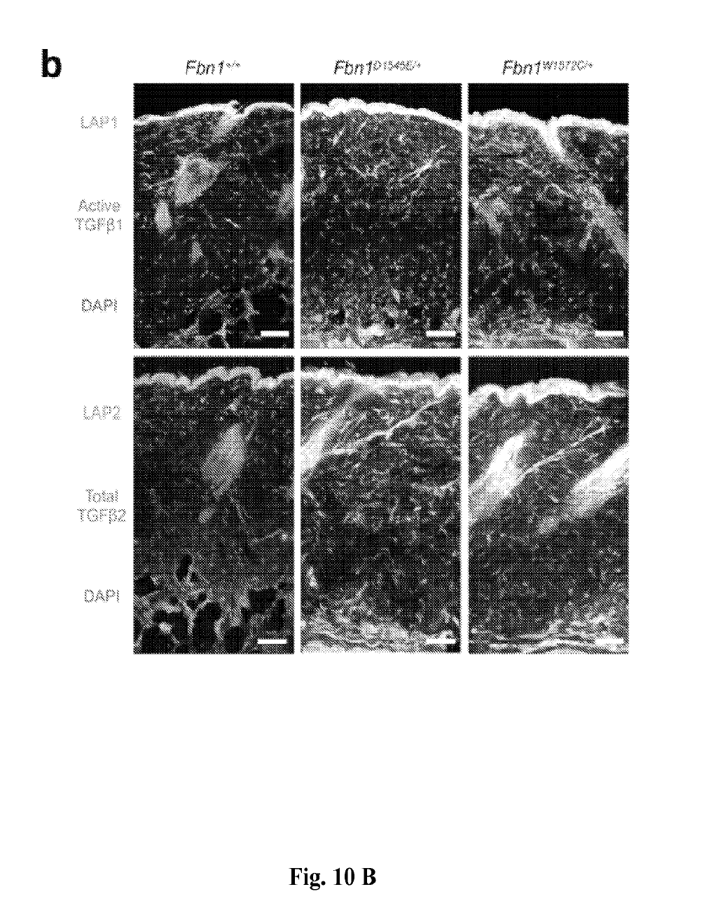

To address this, we used flow cytometry to monitor mutant mice for increased cell surface expression of the 3 integrin subtypes (αvβ5, αvβ6, αvβ8) known to support potent TGFβ activation [6]; this was not observed (FIG. 10A). In addition, immunofluorescence analysis of skin in mutant mice did not reveal increased expression of free TGFβ1 (FIG. 10B), which is known to be activated by integrins through interaction with the RGD sequence in its LAP (LAP1). There was an increase in total (free and active) TGFβ2 (FIG. 10B), which has not been demonstrated to be activated by integrins (presumably due to the absence of an RGD sequence in LAP2) [6]. Furthermore, there was excessive concentration of both LAP1 and LAP2 in the dermis of mouse models of SSS, suggesting accumulation of the LLC for TGFβ1 and TGFβ2, respectively. While we cannot exclude a contribution of integrinmediated TGFβ activation, these data suggest that enhanced TGFβ bioavailability prominently contributes to increased TGFβ activity in mutant mice.

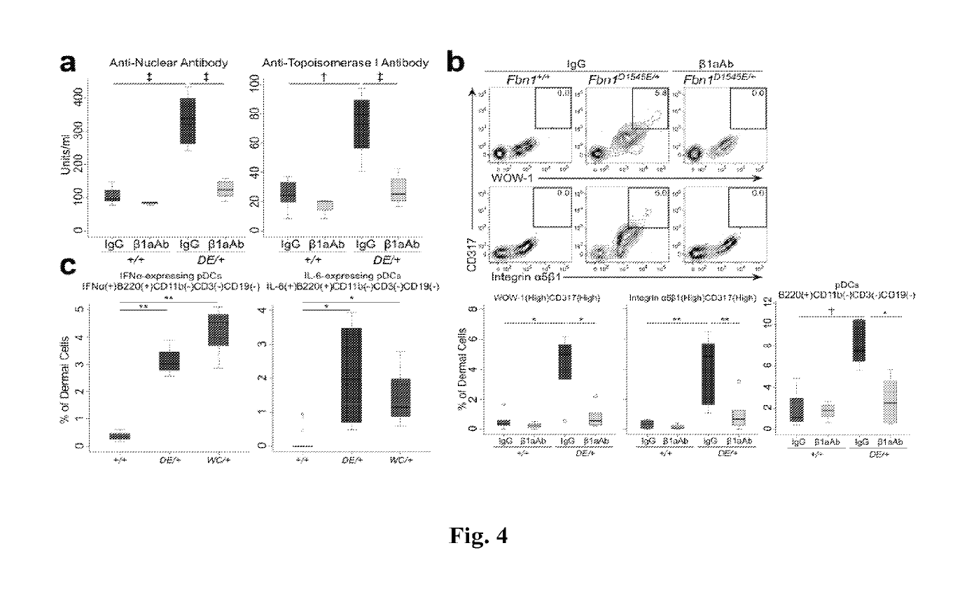

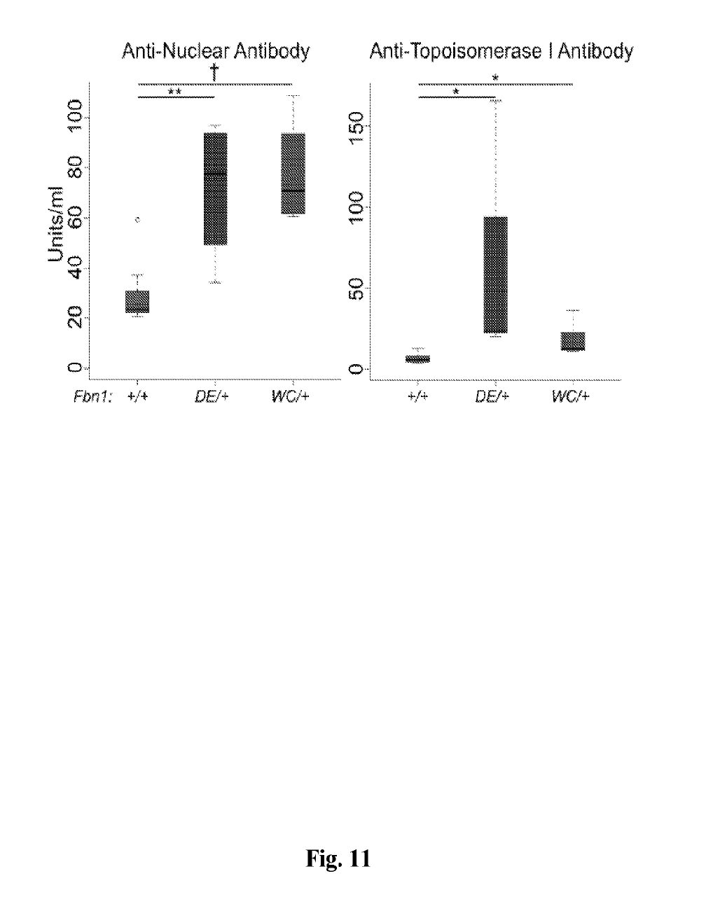

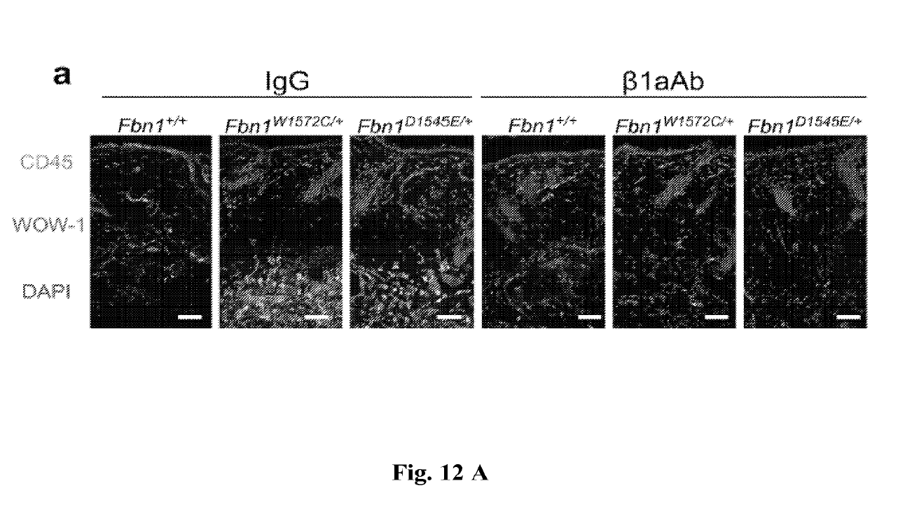

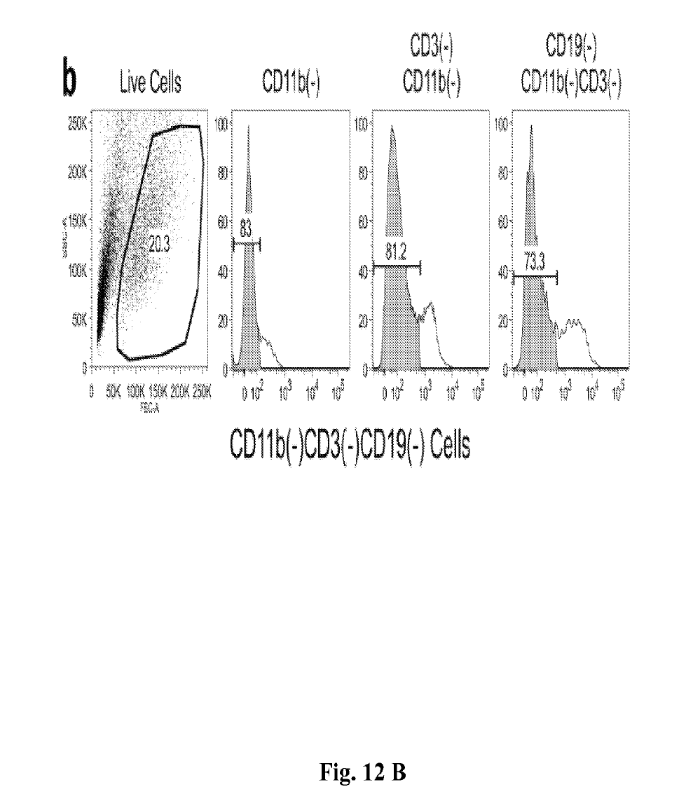

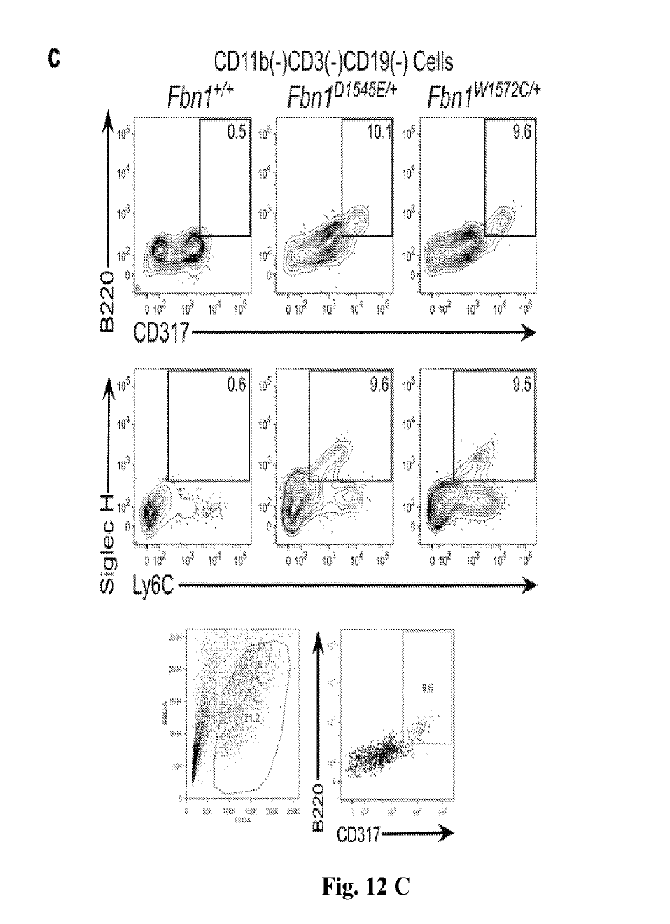

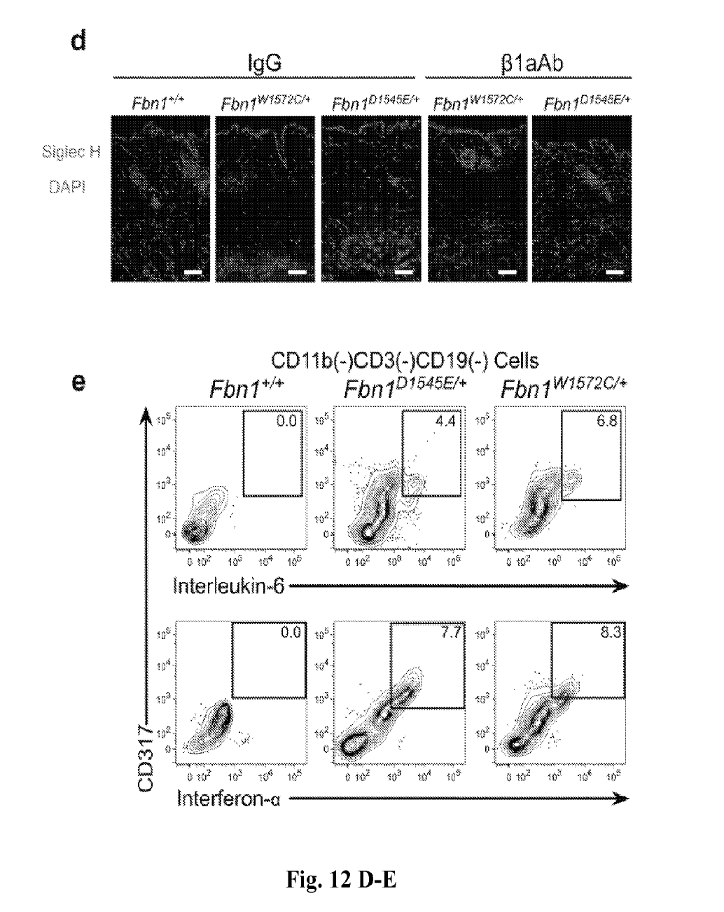

As seen in SSc, SSS mouse models show circulating anti-nuclear and antitopoisomerase I antibodies (FIGS. 4A and 11). The finding that the deep dermal fibrosis seen in early SSS (FIG. 1) co-localizes with high expression of active β3 integrin (as measured by WOW-1) and accumulation of CD45(+) marrow-derived cells (FIG. 12A) prompted speculation that an infiltrating class of immune cells might contribute to disease progression. In keeping with this hypothesis, nearly all dermal cells expressing high levels of α5β1 and active β3 integrins in SSS mice are CD317(+) plasmacytoid dendritic cells (pDCs) (FIG. 4B). The finding that SSS mice show dermal enrichment for cells that are CD11b(−) CD3(−) CD19(−), but B220(+), Siglec H (+), and Ly6C(high) further validated this identity (FIGS. 4B, 12B-D) [9]. As is characteristic for mature and active pDCs, these dermal cells express the pro-inflammatory cytokines interleukin (IL)-6 and interferon (IFN)-α in SSS (FIGS. 4C, 12E) [9].

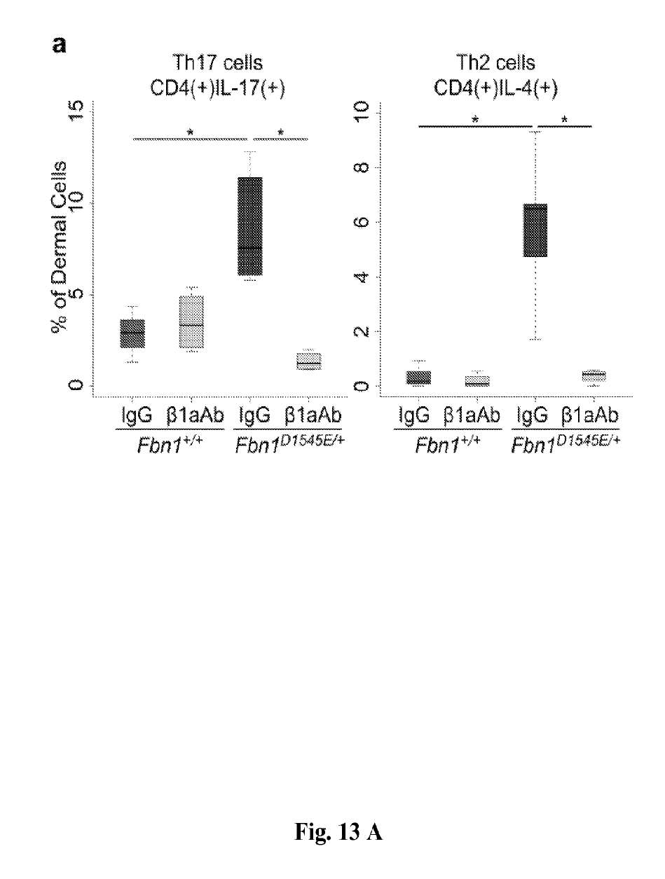

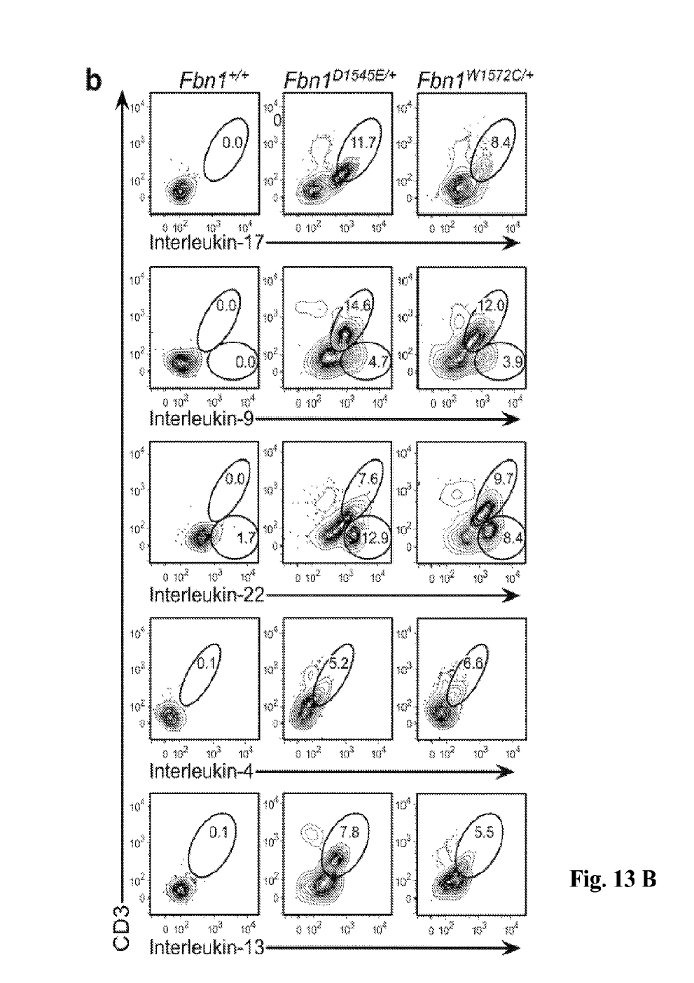

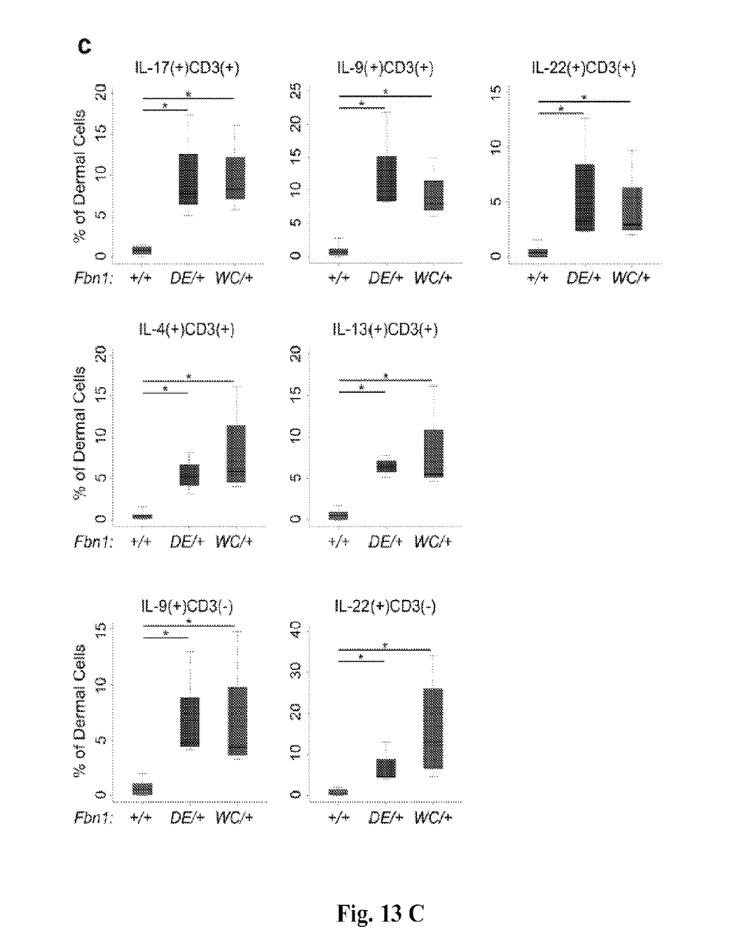

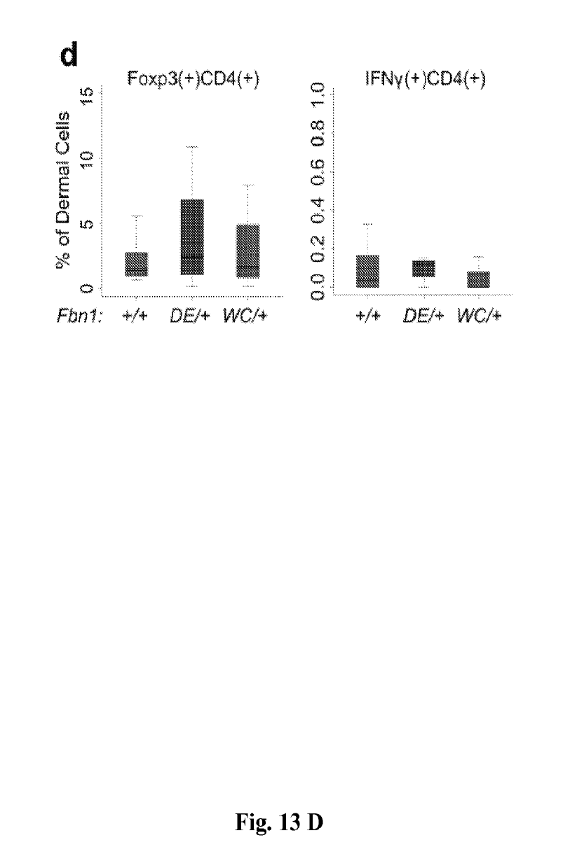

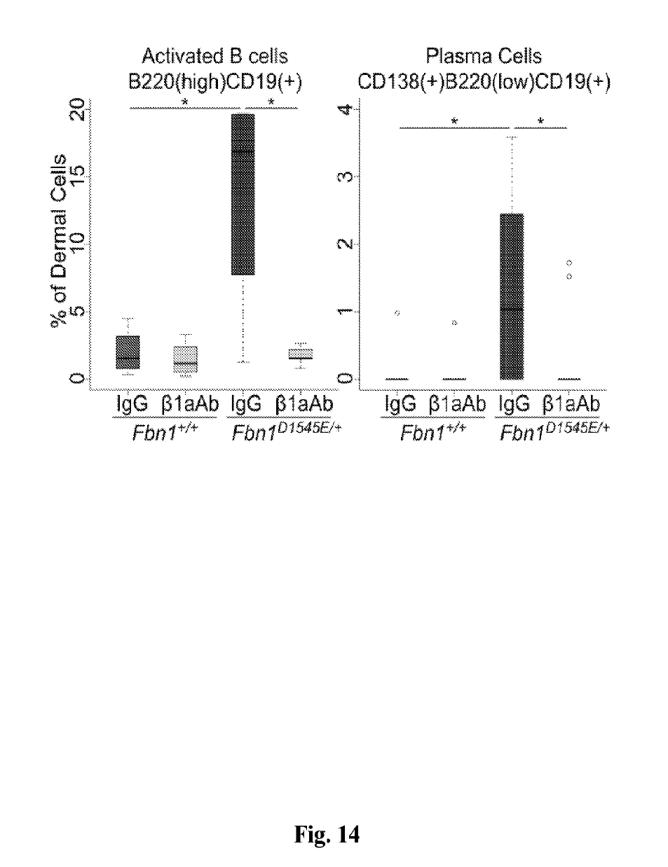

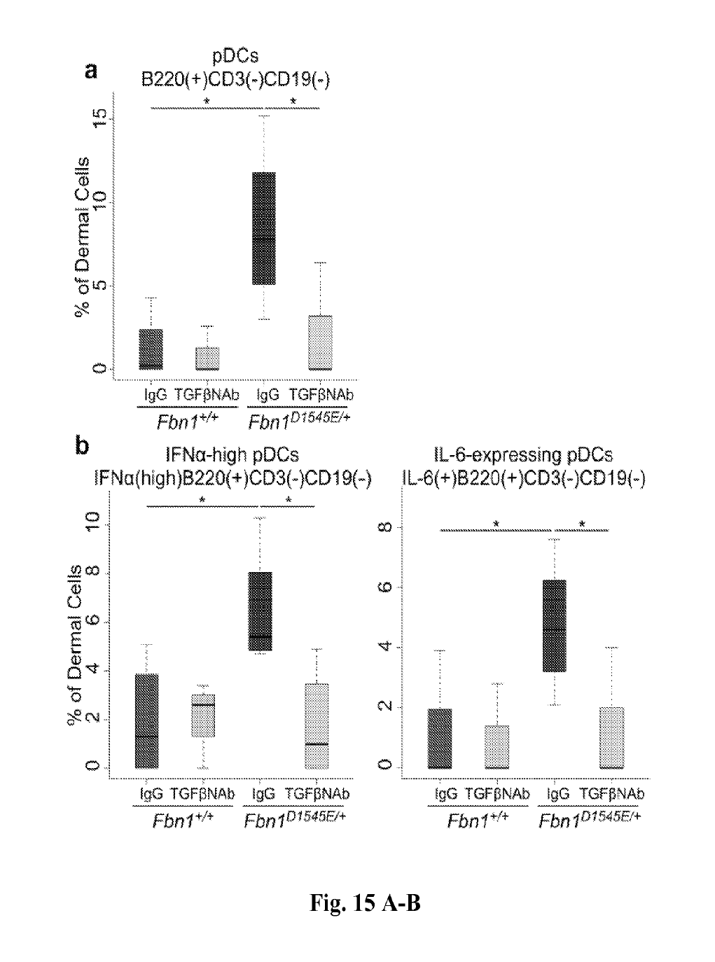

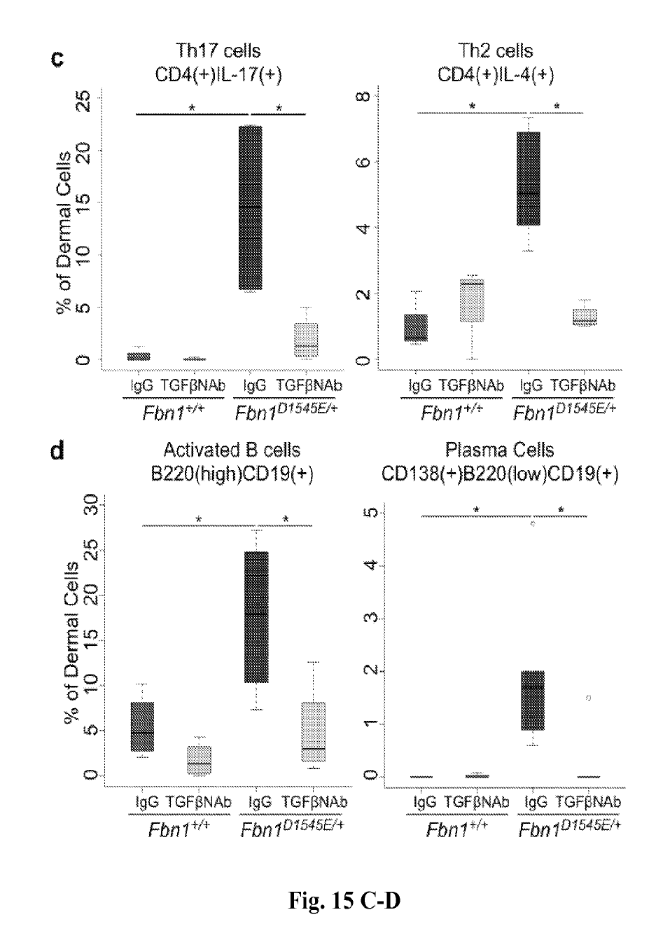

There is also evident polarization toward pro-inflammatory T helper (Th) cell populations in the skin of SSS mice, with accumulation of CD4(+)IL-4(+) Th2, CD4(+)IL-17(+) Th17 cells, and CD4(+)IL-9(+) Th9 cells (FIG. 13A). In keeping with Th2, Th9, and/or Th17-skewing, there was also increased expression of IL-9, IL-13, and IL-22 by CD3(+) cells in the dermis of SSS mice (FIG. 13A-C). There was no corresponding increase in either IFNγ(+)CD4(+) Th1 or FoxP3(+)CD4(+) Tregulatory (Treg) cells in mutant animals (FIG. 13D). Finally, the dermis of SSS mice also shows infiltration with B220(high)CD19(+) activated B cells and CD138(+)B220(low)CD19(+) plasma cells (FIG. 14). These abnormalities, including circulating autoantibodies and immune cell infiltration/activation, were normalized upon treatment of mutant mice with β1aAb (FIGS. 4, 13, and 14). A similar response was seen in association with reversal of skin fibrosis upon treatment with TGFβNAb (FIG. 15).

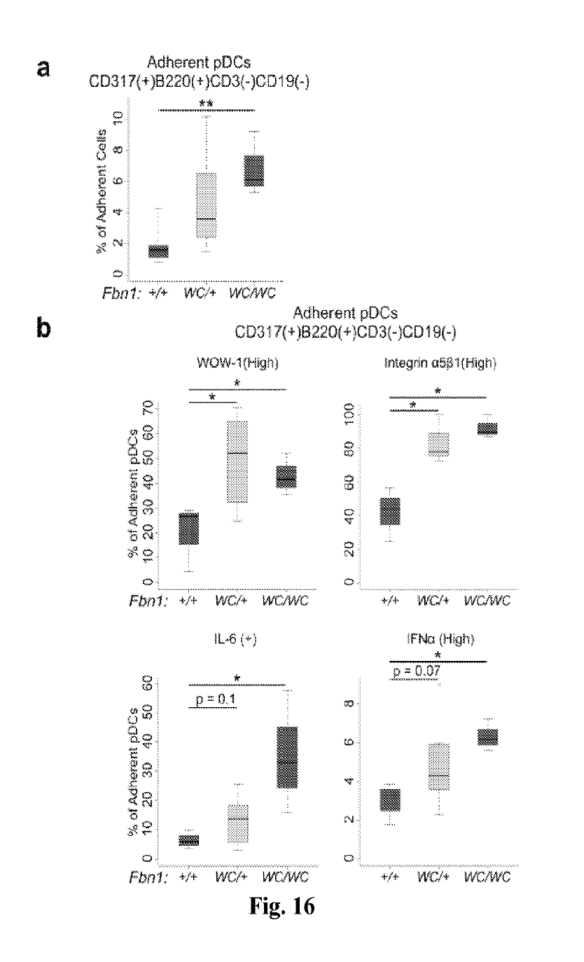

We hypothesized that altered presentation of the fibrillin-1 RGD sequence might directly influence integrin expression by and the performance of pDCs. In keeping with this hypothesis, we found that wild-type spleen derived pre-pDCs showed increased adherence and activation (IFN-α and IL-6 expression) when plated on the matrix expressed by SSS murine embryonic fibroblasts (MEFs) as compared to control MEFs (FIG. 16).

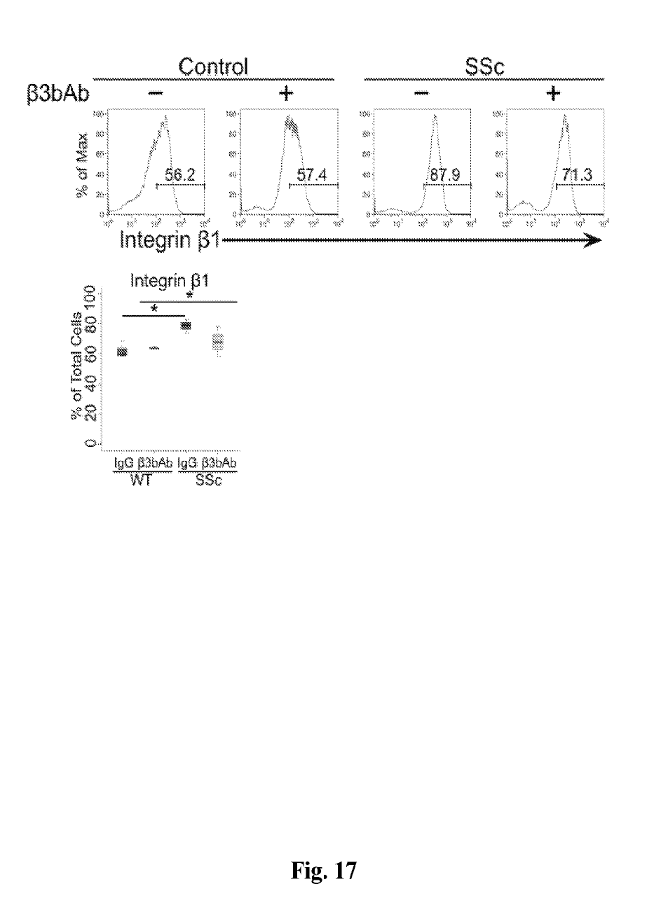

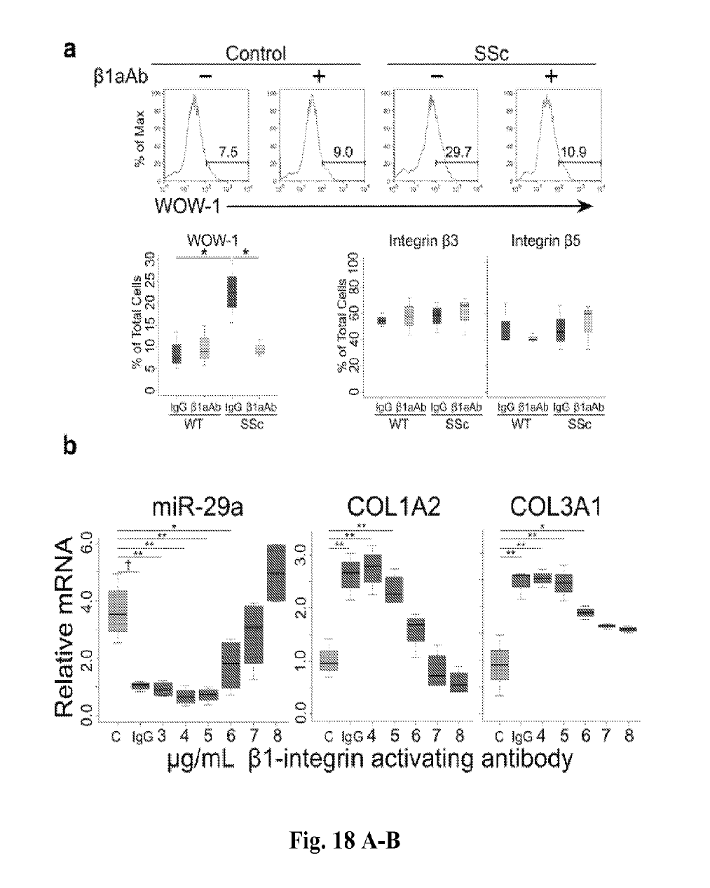

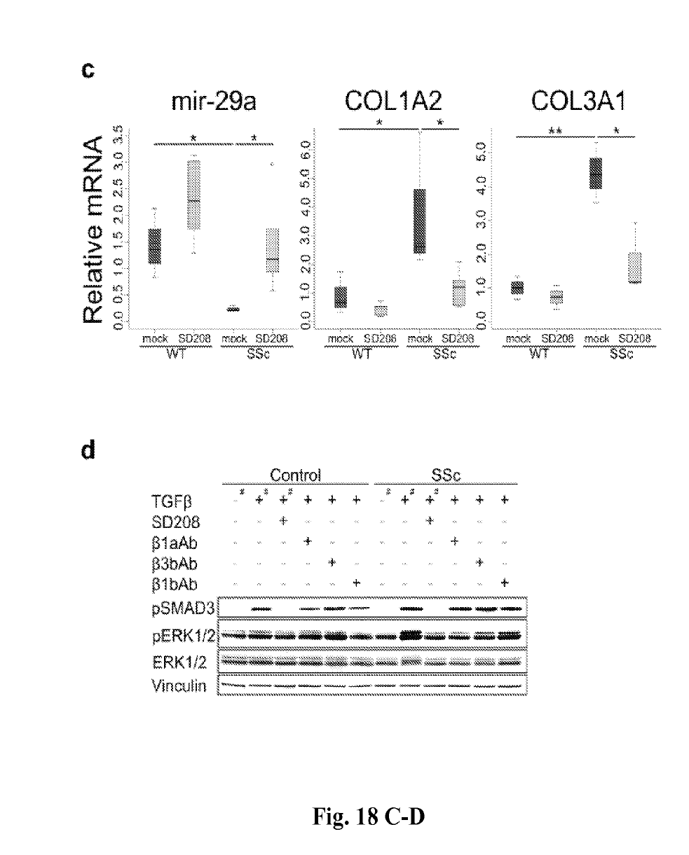

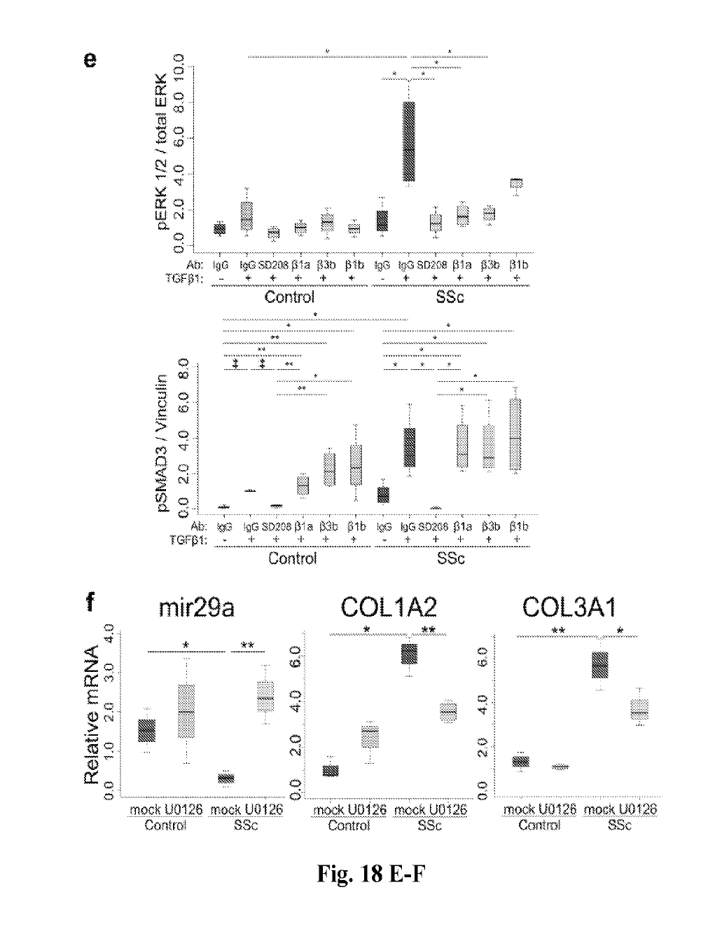

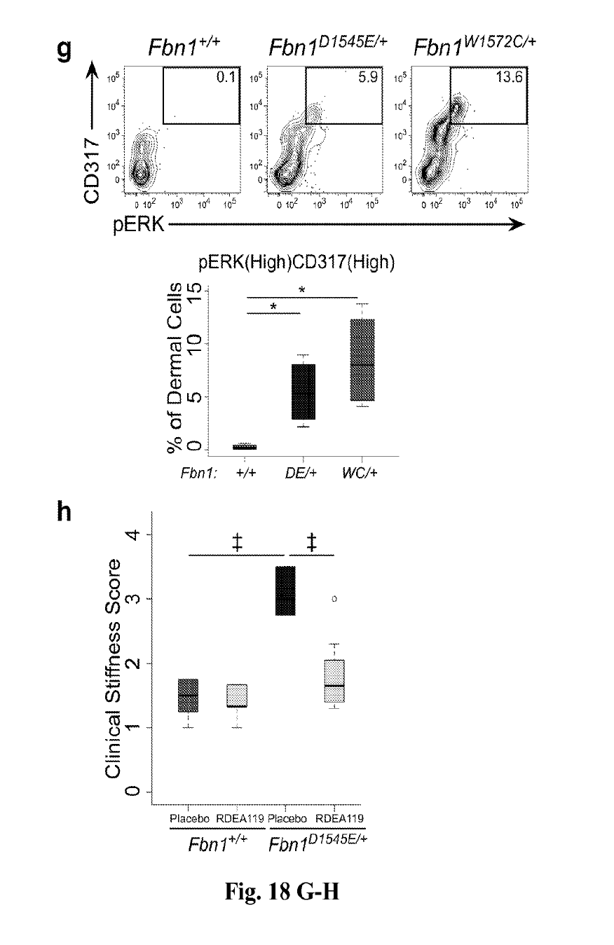

SSc fibroblasts demonstrated increased cell-surface presentation of total β1 integrin (FIG. 17) and active β3 integrin (as monitored by WOW-1 staining) in comparison to controls, whereas levels of total β3 and β5 integrins were normal (FIG. 18A). Treatment with β1aAb TS2/16, which promotes and stabilizes integrin β1-ligand interactions, normalized active β3 integrin cell-surface levels (FIG. 18A). Treatment with β3 integrin-blocking antibody (β3bAb) did not significantly reduce cell-surface presentation of total β1 integrin (FIG. 17). Human SSc cells in culture showed decreased levels of microRNA-29 (miR-29) (FIG. 18B), a small regulatory RNA that is repressed by TGFβ and is known to inhibit expression of multiple matrix elements (including types I and III collagen) and to suppress fibrosis in selected disease states [10,11]. Treatment with β1aAb normalized miR-29 expression and attenuated expression of types I and III collagen in SSc fibroblasts in a dose-dependent manner (FIG. 18B). SD208, an antagonist of the kinase activity of the type I TGFβ receptor subunit (TβRI), also normalized collagen and miR-29a expression (FIG. 18C).

In addition to canonical (Smad-dependent) signaling, TGFβ can also initiate socalled noncanonical cascades, prominently including extracellular signal regulated kinase (ERK1/2) [5]. SSc fibroblasts showed normal Smad3 phosphorylation (pSmad3) in response to stimulation with TGFβ1 that was not influenced by integrin-modulating therapies, but uniquely showed TGFβ1-dependent phosphorylation of ERK1/2 (pERK1/2), when compared to control fibroblasts, that was normalized upon treatment with either β1aAb or β3bAb (FIGS. 18D and 18E). The activation of ERK1/2 in SSc fibroblasts was seen within 5 minutes of TGFβ1 stimulation and was inhibited by pretreatment with SD208, suggesting a relatively direct response (FIGS. 18 D and 18E). In keeping with a pathogenic contribution of pERK1/2, treatment of SSc fibroblasts with U0126, an inhibitor of the mitogen-activated protein kinase/ERK kinase (MEK), increased miR-29a levels and reduced collagen expression in SSc fibroblasts (FIG. 18F). Both SSS mouse models show excessive activation of ERK1/2 in CD317(+) pDCs and other dermal cells (FIG. 18G). Treatment of Fbn1^{D1545E/+} mice with the MEK-inhibitor RDEA119 prevented skin stiffness, dermal collagen accumulation, and loss of subcutaneous fat (FIG. 18H,I).

This study shows that point mutations specifically in the sole integrin-binding domain of fibrillin-1 are sufficient to recapitulate the SSS phenotype in mice and to initiate many findings reminiscent of SSc including dermal fibrosis, autoantibody production, high IFN-α expression, Th2 and Th17 polarization, and accumulation of activated B cells and plasma cells in the skin [1, 2, 4, 12, 13]. While prior studies have reported autoantibodies and subdermal fibrosis in tight skin (Tsk) mice harboring a large central duplication in Fbn1, there are no direct human correlates and both the mechanism and pathogenic relevance remain unclear [14, 15]. In SSS, all of these processes can be functionally linked to altered integrin expression and/or function since they are prevented by integrinmodulating therapies (activation of β1 integrin or genetic targeting of β3 integrin). While skin fibrosis was observed in mice upon conditional silencing of β1 integrin expression in keratinocytes [16], targeting of Itgb1 in fibroblasts afforded relative protection against bleomycin-induced skin fibrosis [17]. This apparent discrepancy has not been mechanistically explained.

A comparison of MFS and SSS highlights the complicated role of the extracellular matrix in cytokine regulation. Unlike MFS, where a deficiency of extracellular fibrillin-1 is seen, SSS-specific FBN1 mutations promote increased deposition of abnormal microfibrillar aggregates that fail to make contact with neighboring cells but retain the ability to bind to the TGFβ LLC. This results in decreased or increased concentration of latent TGFβ in tissues in MFS or SSS, respectively [3, 5].

In MFS, it is posited that decreased LLC concentration is offset by increased TGFβ activation, but that this may occur in a tissue-specific manner (e.g., in the lung and aorta) [5,6]. The relative deficiency of microfibrils and hence latent TGFβ in MFS would mandate ongoing TGFβ production to support high signaling, whereas the high dermal concentration of TGFβ in SSS might allow a more sustained enhanced signaling state. Curiously, this does not appear to occur in all tissues where fibrillin-1 is expressed, perhaps due to different repertoires of expressed integrin subtypes that vary in their sensitivity to conformational changes induced by SSS mutations and/or tissue-specific differences in the regulation of microfibrillar assembly.

The stiffened ECM in SSS could support mechanical traction-based activation of the excessive amounts of latent TGFβ in the dermis, a plausible feed-forward mechanism for the observed fibrosis [8]. Thus the level of TGFβ signaling in a given tissue may, at least in part, be determined by integration of both positive and negative regulation by microfibrils [5,6]. Although the cause remains unknown, the skin from patients with active diffuse SSc also shows aberrant and excessive microfibrillar aggregates that retain the ability to concentrate latent TGFβ [3].

While the cell type that first detects and responds to aberrant presentation of the RGD sequence in fibrillin-1 remains unknown, it is interesting to speculate involvement of pre-pDCs that normally perform a surveillance function for viral pathogens at low concentrations in the skin. Prior work has shown that α5β1 integrin influences DC adhesion, migration, and maturation, and that migration is inhibited by β1aAb, at least in part through podosome disassembly [18]. Furthermore, a specific role for α5β1 integrin in pre-pDC chemotaxis and trafficking has been demonstrated [19]. It is therefore evident that pre-pDCs are informed by and respond to their matrix environment, with fibrillin-1 potentially serving as a prominent informant. In keeping with this, our in vitro observations (FIG. 16) suggest that an altered matrix environment, devoid of any systemic influence, is sufficient to promote pDC recruitment and activation. Whether this relates to loss of a physiologic inhibitory signal by normal microfibrils or a pathogenic gain-of-function by the abnormal microfibrillar aggregates seen in SSS and SSc remains to be determined.

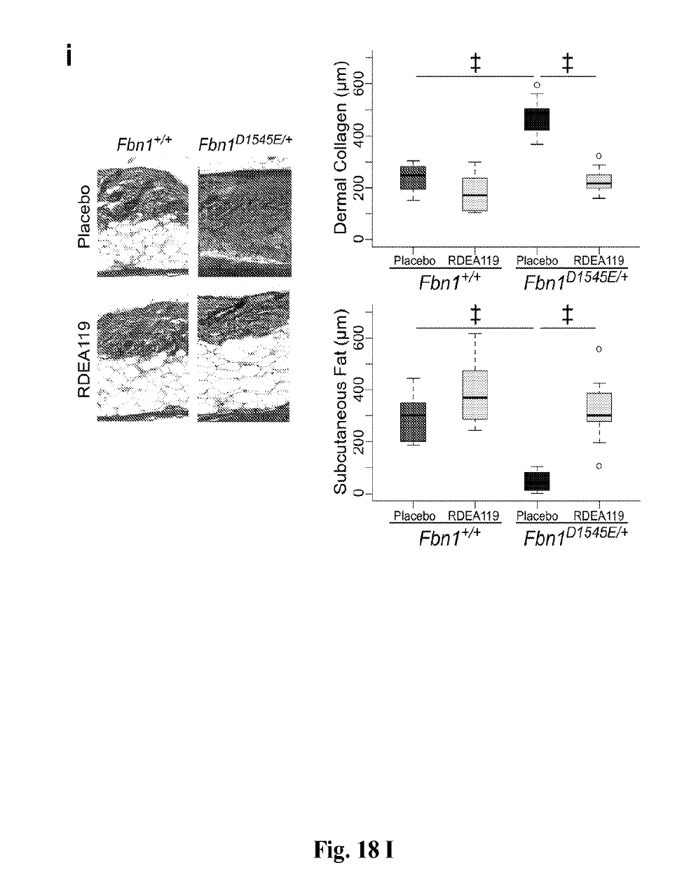

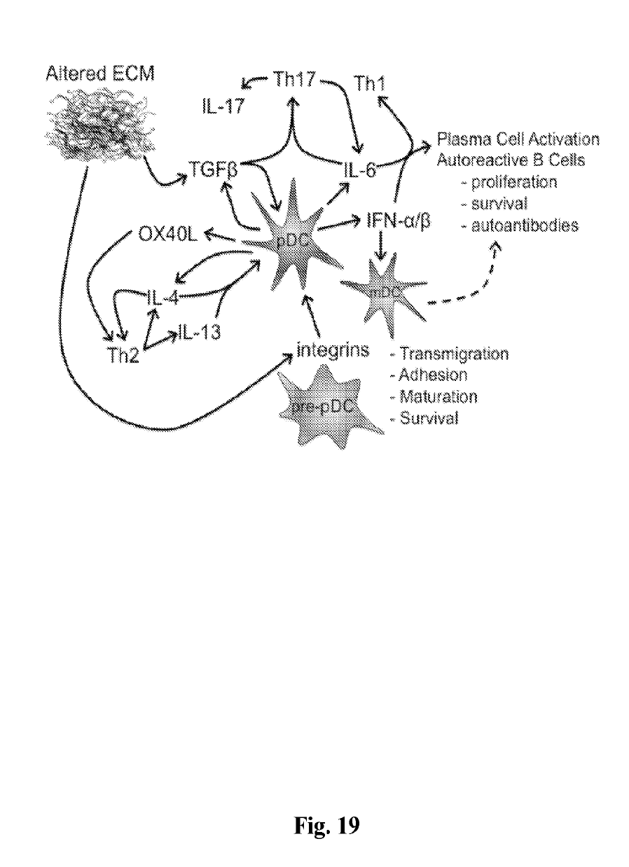

pDCs are a major source of IFN-α and are capable of inducing Th2- and Th17-skewing, autoreactive B cell and plasma cell differentiation, and autoantibody production (FIG. 19) [9,12,20-22]. Plasmacytoid dendritic cells have also previously been implicated in multiple autoimmune processes (including SSc) [9,12,20-22]. Although pDCs can contribute to both tolerogenic Treg or autoinflammatory Th17 cell commitment, in vitro experiments suggest that TGFβ-treated pDCs favor the latter via a Smad dependent mechanism [23]. While the altered matrix environment in SSS likely contributes to excessive TGFβ activity early in the course of disease, TGFβ induces its own production and activation by pDCs, as well as IL-6 secretion (known prerequisites for Th17 polarization) [23]. pDCs can also induce either Th1 or Th2 skewing via IL-6/IFN-α- or OX40L/IL-4-dependent mechanisms, respectively (FIG. 19) [9]. pDCs in a Th2 environment become activated and show enhanced IL-4 secretion, constituting a potential feed-forward mechanism for maintenance of a Th2 response [24]. In the context of high TGFβ-signaling, this might also allow for Th9-skewing, given that IL-4 and TGFβ are known to drive Th9 differentiation [25]. Th2-, Th17- and pDC related cytokines, including IL-4, IL-6, IL-13, IL-17 and IFN-α, have been prominently implicated in the fibrotic response in diverse disease states, including SSc [1, 2, 4, 9, 12, 13, 20-22]. To our knowledge, this is the first study that implicates TGFβ in pDC recruitment.

While many studies have highlighted the contribution of integrins to fibrotic disease [8], the focus has been on the ability of certain integrins to release (activate) TGFβ1 or TGFβ3 from the LLC through a direct interaction with RGD sequences in LAP1 and LAP3 [8]. Multiple observations in this study suggest that enhanced TGFβ bioavailability, rather than activation, may be the primary determinant of increased TGFβ activity in SSS and perhaps SSc. Our in vitro data in SSc fibroblasts suggest that cell surface integrins can influence the inherent signaling properties of the TGFβ receptor complex in response to free and active TGFβ. While the initiating pathogenic event in SSc remains unknown, this study provides evidence for a cell autonomous signaling defect. In theory, this could relate to primary but poorly penetrant genetic alterations or fixed epigenetic modifications, both of which may require a major environmental trigger.

Activation of ERK1/2 has previously been implicated in the TGFβ-mediated fibrotic response in general and specifically in SSc fibroblasts [26-28]. Asano and colleagues previously observed that constitutive ERK1/2 signaling in SSc fibroblasts drives expression of integrin αvβ3. Both αvβ3 and TGFβ were required for excessive collagen production [28]. Despite overlapping observations and the common conclusion that αvβ3 represents an attractive therapeutic target, this study places ERK1/2 activation downstream of both TGFβ and enhanced active αvβ3 expression in SSc fibroblasts and uniquely shows phenotypic rescue upon ERK antagonism in an in vivo model of scleroderma. Furthermore, we show prominent ERK1/2 signaling in pDCs in SSS mice, a described prerequisite for the stabilization, nuclear export and translation of IFN-α mRNA [29], and for toll-like receptor-mediated expression of inflammatory cytokines [30]. While prior work associated low levels of miR-29, a negative regulator of collagen expression, with fibrotic diseases including post-injury cardiac fibrosis [10] and SSc [11], this study is the first to offer a pathogenic sequence for scleroderma that integrates structural matrix elements, cell-surface integrins, TGFβ signaling, ERK activation, and miR-29.

SSS mouse models demonstrate the potential to reverse established dermal fibrosis, suggesting several potential therapeutic strategies for the treatment of skin fibrosis, including β1 integrin activation and blockade of β3 integrin, TGFβ or ERK signaling. These findings affirm the relevance of studying a rare but tractable Mendelian form of scleroderma to the understanding of more common but complex presentations of fibrotic skin disease. When paired with the ability to perform pre-clinical trials in the first described mouse models of a genetically defined human presentation of scleroderma, the potential for therapeutic advancement seems promising.

Methods.

Subjects.

Patients were recruited from the Scleroderma Center and Connective Tissue Clinic at Johns Hopkins Hospital (F.M.W. and H.C.D.). All skin biopsies and research protocols were performed in compliance with the Johns Hopkins School of Medicine Institutional Review Board and after informed consent.

Mice.

All mice were cared for under strict compliance with the Animal Care and Use Committee of the Johns Hopkins University School of Medicine. Fbn1^{D1545E/+} and Fbn1^{W1572C/+} mice were generated by homologous recombination as described in the next section. Itgb3+/− mice were purchased through Jackson Laboratories (Bar Harbor, Me.) as heterozygotes. All experimental mice were on a mixed C57B1/6J and 129/SvEv background. To minimize potentially confounding background effects, all comparisons between genotypes and between treatment arms within a genotype where made between sex-matched littermates.

Generation of Fbn1^{D1545E/+} and Fbn1^{W1572C/+} Mice.

Fbn1^{D1545E/+} and Fbn1^{W1572C/+} mice were generated by homologous recombination (FIG. 5A). A 10 kb Fbn1 fragment was generated by PCR from mouse genomic tail DNA, digested with Acc65 and NheI restriction enzymes (NEB), and ligated into pSL301 (Invitrogen Corp.). Site-directed mutagenesis (SDM) was performed using the QuikChange mutagenesis kit (Stratagene Inc.), creating either the D1545E or W1572C mutation. The targeting vector was assessed by sequence analysis. SDM was again performed to remove the AatII restriction site from pSL301. The NeoR cassette was amplified from pEGFP-C1 (Invitrogen Corp.) and the amplicon was subcloned into pCR2.1-TOPO (Invitrogen Corp.). A fragment containing the AatII restriction site and NeoR, with flanking loxP sequences, was subcloned into a unique AatII site in the Fbn1 intron before exon 38. The sequences of the loxP sites and SDM-created mutations were confirmed by direct sequencing. The vector was linearized using a unique (NruI) site and electroporated into R1 ES cells. Positive clones were identified by Southern blot analysis (FIG. 5B) as previously described [31].

Positive clones were injected into 129/SvEv blastocysts at ED 3.5 and transferred into pseudopregnant females. Chimeric offspring were mated to C57B16/J mice, and germline transmission was observed for at least three independent targeting events for each genotype. All exons encompassed by and immediately flanking the targeting vector were analyzed by sequencing of PCR amplified genomic DNA derived from mutant animals to demonstrate the fidelity of targeting. Complete concordance of phenotype for three or two independent lines for mutations W1572C or D1545E, respectively, excluded any major offtarget effect. Mice were genotyped on the basis of creation of a new AciI site (W1572C) or destruction of a BsmAI site (D1545E) in correctly targeted mice (FIG. 5C). Primers used for amplification: Sense: 5′-GATCCCACCACCTGCATC-3′(SEQ ID NO:1); Antisense: 5′-CATGTGTTCACAGAAGGACAC-3′ (SEQ ID NO:2). The loxP-flanked NeoR was removed by breeding Fbn1^{D1545E/+} and Fbn1^{W1572C/+} mice with transgenic mice that ubiquitously expresses Crerecombinase using a EIIa-promoter, purchased through Jackson Laboratories (Bar Harbor, Me.). Over 85 embryos were genotyped at ED 10.5 for Fbn1^{D1545E/+} homozygosity.

In Vivo Drug Treatment.

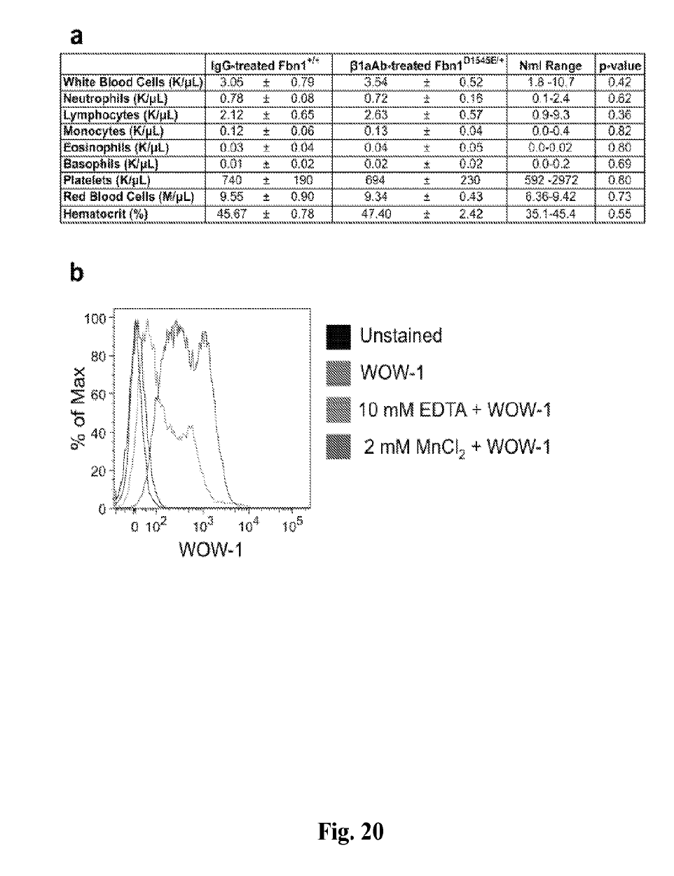

All antibodies used to treat mice or cells were azide-free. Male mice were treated with β1 integrin activating antibody (β1aAb, Rat Clone 9EG7, BD Biosciences, special-ordered >98% pure and azide-free) or an isotype-matched control (Rat IgG2a, κ, special-ordered >98% pure and azide-free, BD Biosciences) by intraperitoneal injection at 2 mg/kg every five days for twelve weeks, beginning at one month of age. Complete blood cell counts were performed to exclude pancytopenia in β1aAb-treated animals (FIG. 20A). For the TGFβ-Neutralizing trial, three-month-old male mice were treated with pan-specific TGFβ-Neutralizing antibody (Mouse Clone 1D11, catalog #MAB1835, R&D) or an isotype control (Mouse IgG1, Clone 11711, cat#MAB002, R&D) by intraperitoneal injection at 10 mg/kg every other day for twelve weeks.

RDEA119 was generously provided by Craig J. Thomas, Samarjit Patnaik, and Juan J. Marugan (National Institutes of Health Chemical Genomics Center, Rockville, Md., USA). RDEA119 was reconstituted in 10% 2-hydroxypropyl-betacyclodextrin (Sigma-Aldrich) dissolved in PBS, and administered twice daily by oral gavage at a dose of 25 mg/kg. Treatment was initiated at 1 month of age and continued for 8 weeks. 10% 2-hydroxypropyl-beta-cyclodextrin dissolved in PBS was administered as a control. Given the absolute concordance regarding pathology and therapeutic responses for Fbn1^{D1545E/+} and Fbn1^{W1572C/+} mice seen early in this study, later studies focused on Fbn1^{D1545E/+} mice to limit the expense associated with in vivo antibody (TGFβNAb and β1aAb) and drug (RDEA119) trials.

Stiffness Scoring.

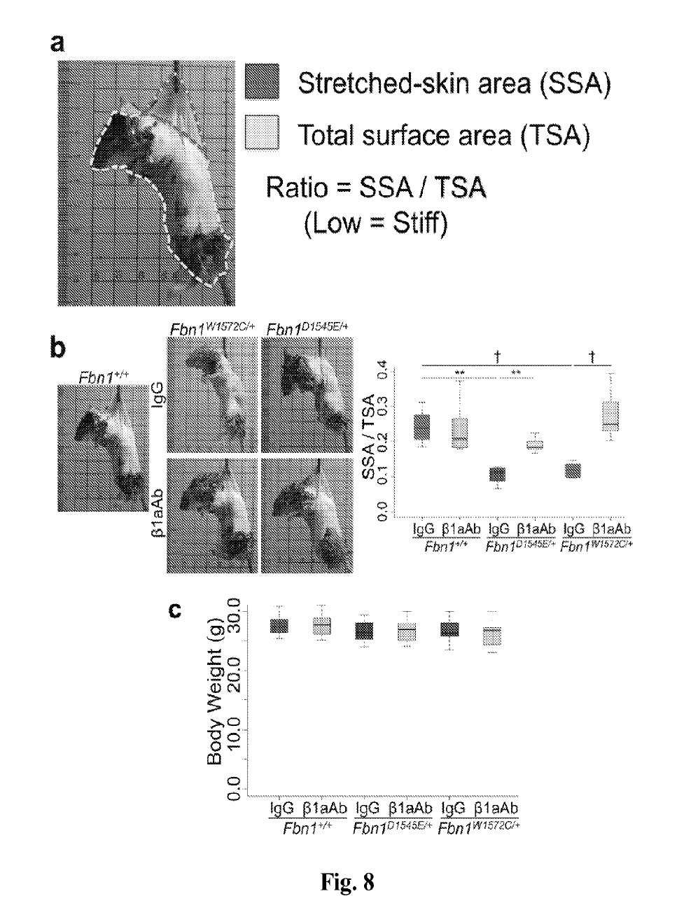

A clinical stiffness score was assigned by five blinded observers. Observers were blinded to genotype and treatment status. Mice were assessed in random order. A score of 1 indicates no stiffness (i.e. identical to wild-type mice). A score 4 indicates extreme stiffness based upon prior experience with untreated SSS mice, with 2 and 3 indicating a subjective assessment of an intermediate level of stiffness. Early in the course of studies, the same mice were assessed by the same observer on a different day. This pilot demonstrated excellent intraobserver concordance. To measure stretched skin area (SSA) and total surface area (TSA), mice were anesthetized with isofluorane and the back skin was shaved and briefly treated with Nair® cream. Area measurements were performed with NIH image J software (National Institute of Health, Bethesda, Md., USA). Mice were then briefly suspended with forceps secured to the back skin by a clamp and photographed in profile in a uniform manner (FIG. 8A,B). There were no differences in body weight between all experimental groups (FIG. 8C).

Histology.

For tissue analysis, animals were euthanized through inhalational halothane (Sigma) or anesthetized with isofluorane. Back skin was shaved and briefly treated with Nair® cream before biopsy. Fixed skin was paraffin-embedded, sectioned, and stained with a standard Masson's trichrome stain. Dermal and subcutaneous fat thickness was measured using high-powered fields as described previously [32]. Immunofluorescent staining was performed on frozen sections as previously described [33]. Active αvβ3 was detected using the WOW-1 antibody (a gift from Dr. Sanford Shattil [39]) and an anti-mouse Alexa Fluor-594 F(ab′)2 fragment secondary (Invitrogen cat#A11020).

Other antibodies used include Anti-CD45 antibody (BD, cat#550539), anti-Siglec H (ebiosciences cat#14-0333-81) and antibodies to LAP1 (cat#141402, BioLegend), LAP2 (cat#LSC137100, Lifespan BioSciences) active TGF(1 (Clone LC(1-30), a gift from Kathleen Flanders), and total TGFβ2 (cat#ab66045, abcam). With the exception of WOW-1, all other antibodies were conjugated via an amine-based Alexa Fluor antibody labeling kit (Invitrogen, cat#A-20181, A20187, A-20185, A-20186).

Electron Microscopy.

Electron microscopy (EM) was performed as previously described [34].

Enzyme-Linked Immunosorbent Assay.

Mouse sera was collected and enzyme-linked immunosorbent assays (ELISAs) were performed using the Mouse Anti-Nuclear Antigens and Mouse Anti-Scl70kits (cat#5210 and 6110, AlphaDiagnostic) according to the manufacturer's instructions.

Cell Culture.

Primary human dermal fibroblasts (HDFs) were derived from skin biopsies from 5 patients with active diffuse systemic sclerosis and 6 healthy controls. Biopsies were taken from the forearm and cultured as previously described [6]. All experiments were performed in cell lines at low (<5) passage. Primary mouse embryonic fibroblasts (MEFs) were derived from E13.5 embryos as described previously [7]. Murine plasmacytoid dendritic cells (pDCs) were isolated from the spleens of wild-type C57B16/J mice using the Plasmacytoid Dendritic Cell Isolation Kit II (cat#130-092-786, Miltenyi Biotec) and a midiMACS™ Separator (cat#130-042-302, Miltenyi Biotec) according to the manufacturer's instructions. The pDC-containing cell suspensions routinely had greater than 95% purity, as detected by flow cytometry.

For MEF/pDC co-culture experiments, MEFs were cultured to complete confluency in culture medium containing RPMI-1640, streptomycin 100 μg/ml, penicillin 100 U/ml, 2 mM L-glutamine (Gibco®) and 10% heat-inactivated fetal calf serum. 72 hours post-confluence, 5×106 murine splenic pDCs were plated onto MEF monolayers. After 72 hours of co-culture, both adherent and non-adherent cellular fractions were harvested, counted, and analyzed by flow cytometry.

Flow Cytometric Analysis.

Mouse skin was digested for flow cytometric analysis as previously described [35]. On average, 4×106 cells were obtained from a 1×2 cm2 piece of skin for wildtype mice, and 8×106 cells were obtained from either SSS mouse model. Murine Fc receptors were blocked using Abs against mouse CD16/32 antigens (cat#553141, BD Biosciences). Murine plasmacytoid dendritic cells were isolated as previously reported [38]. All isolated cells (including murine dermal cells, cultured MEFs, splenic murine pDCs, or human dermal fibroblasts) were stained and fixed using the BD Cytofix/Cytoperm™ system (cat#554722, BD Biosciences). Data were acquired using CellQuest-Pro software on a FACSCalibur flow cytometer or BD FACSuite™ software on a FACSVerse flow cytometer (BD Biosciences, San Jose, Calif., USA). Data were analyzed and all flow cytometry plots were contour plots (with outliers) that were generated with FlowJo® software (TreeStar).

For histograms, FlowJo software divides all events into 256 “bins,” which are numerical ranges for the parameter on the x-axis. The percent of maximum (yaxis) is the number of cells in each bin divided by the number of cells in the bin that contains the largest number of cells. Gating for live cells was based on staining with the LIVE/DEAD® Fixable Dead Cell Stain Kit (Invitrogen, cat#L34955). All staining was performed with fluorophore-conjugated primary and isotype control antibodies. All antibodies were either purchased as fluorochrome conjugates or conjugated via amine-based Alexa Fluor antibody labeling kits (cat#A-20181, A20187, A-20185, A-20186, Invitrogen). Mouse and human active αvβ3 was detected fluorophore-conjugated WOW-1 antibody (a gift from Dr. Sanford Shattil). 10 mM Ethylenediaminetetraacetic acid (EDTA) and 2 mM MnCl2 were used as negative and positive controls for αvβ3 activation in flow cytometry experiments (FIG. 20B) [39]. Integrin αvβ5, a subtype known to react with the WOW-1 antibody [9], was monitored in mouse and human cells with a specific antibody (cat#LS-C36943, Lifespan Biosciences).

Other antibodies used on mouse cells were: integrin β1 (Clone eBioHMb1-1, cat#17-0291-80, eBiosciences), integrin β3 (Clone 2C9.G3, cat#12-0611, eBiosciences), integrin α5 (cat#11-0493-83, eBiosciences), integrin 36 (cat#LS-C152915, Lifespan BioSciences), integrin β8 (Clone H-160, cat#sc-25714, Santa Cruz Biotechnology), and pERK1/2 (cat#4370, Cell Signaling). Antibodies used for immunologic characterization of mouse cells from from ebiosciences include IL-13 (cat#53-7133-82) and IL-22 (cat#12-7221-82); from BD biosciences include: Ly6C (cat#560593), CD11b (cat#562127), CD4 (cat#560783), CD8 (cat#560469), CD19 (cat#550992), CD138 (cat#553714), IL-9 (cat#561492), IL-17 (cat#560522), IL-4 (cat#557739), IL-6 (cat#561376), IFN-γ (cat#560660), Foxp3 (cat#560047), and B220 (cat#561226); and from Biolegend® CD3 (cat#100227), Siglec H (cat#129611). The antibody for IFN-α was from PBL interferon source (cat#22100-3). The antibody for CD317 was from eBiosciences (cat#46-3172-82). Antibodies used with human fibroblasts were: integrin β1 (Clone MAR4, cat#557332, BD biosciences) and integrin β3 (Clone VI-PL2, cat#17-0619-42, eBiosciences).

In Vitro TGFβ1-Stimulation of Human Dermal Fibroblasts.

All cells were counted at splitting and all treatments were performed at 70% confluency. Cells were serum starved 48 hours prior to stimulation with 2 ng/mL recombinant TGFβ1 (cat#240-B-010, R&D). When TGFβ1 or vehicle was added, cell culture dishes were immediately rocked on the same rocker three times at 5% CO2, 37° to control for mechanical MAPK activation. Before lysate harvest, cells were washed with pre-warmed (42°) 1×PBS (Gibco®). All antibody treatments of human fibroblasts were added during starvation 48 hours before TGFβ1 stimulation while inhibitors SD208 (1 μM) and UO126 (10 μM) (cat#s 616456 and 662005, EMD Millipore) were added 6 hours prior to stimulation. Antibodies used in vitro were mouse IgG1 (0.2 mg/mL, Clone P3.6.2.8.1, cat#16-4714-81, eBiosciences), IgG2a (0.2 mg/mL, Clone eBM2a, cat#16-4724, eBiosciences), αvβ3-blocking (30 μg/mL, Clone LM609, cat#MAB1976Z, Millipore), β1-activating (7 μg/mL, Clone TS2/16, cat#14-0299, eBiosciences), and β1-blocking (0.2 mg/mL, Clone P4C10, cat#MAB1987Z, Millipore) antibodies.

Western Blotting.

Before lysate harvest, cells were washed with pre-warmed (420) 1×PBS (Gibco®). Total protein was isolated from cells with ice-cold RIPA buffer (25 mM Tris.HCl pH 7.6, 150 mM NaCl, 1% NP-40, 1% sodium deoxycholate, 0.1% SDS) with phosphatase and protease inhibitors (cat#04906837001 and cat#11836170001, Roche). Western blotting was performed using the Bio-Rad and LiCor Odyssey detection systems as previously described [6]. The relative intensities were measured using LiCor Odyssey software. The following antibodies were used: phosphorylated and total ERK (Clone D13.14.4E, cat #4370 and Clone 3A7, cat #9107, Cell Signaling), vinculin (Clone hVIN-1, cat#V9131, Sigma), and phosphorylated and total SMAD3 (cat#1880-1 and 1735-1, Epitomics).

RNA Isolation and qPCR.

Total RNA was isolated from cultured cells or tissue using Trizol (Invitrogen) according to the manufacturer's protocol. Quantitative PCR for miR-29a and 18S rRNA was performed using pre-designed Taqman primers and probes (ABI) according to manufacturer's instructions. Relative quantification for each transcript was obtained by normalizing against 18S transcript abundance according to the formula 2^{-Ct}/2^{-Ct(18S)}.

Statistics and Graphs.

All quantitative data are shown as standard boxplots produced in R statistical software. The upper and lower margins of the box define the 75th and 25th percentiles, respectively; the internal line defines the median, and the whiskers define the range. Statistical analysis was done using two-tailed t-test assuming equal variance between the compared groups (* p<0.05, ** p<0.01, † p<0.001, ‡ p<0.0001). Values outside of the interquartile range (IQR) are shown as open circles (R software-default), but were not excluded from or treated differently in statistical analyses.

### REFERENCES CITED

1. Mayes, M. D., et al. Prevalence, incidence, survival, and disease characteristics of systemic sclerosis in a large US population. Arthritis Rheum. 48, 2246-2255 (2003).
2. Harris, M. L., Rosen, A. Autoimmunity in scleroderma: the origin, pathogenetic role, and clinical significance of autoantibodies. Curr Opin Rheumatol. 15, 778-784 (2003).
3. Loeys, B. L., Gerber, E. E., Riegert-Johnson, D., et al. Mutations in fibrillin-1 cause congenital scleroderma: stiff skin syndrome. Sci Transl Med. 2, 23ra20 (2010).
4. Varga, J., Pasche, B. Transforming growth factor β as a therapeutic target in systemic sclerosis. Nat Rev Rheumatol. 5, 200-206 (2009).
5. Doyle, J. J., Gerber E. E. Matrix-dependent perturbation of TGFβ signaling and disease. FEBS Lett. 586, 2003-15 (2012).
6. Lindsay, M. E., Dietz, H. C. Lessons on the pathogenesis of aneurysm from heritable conditions. Nature. 473, 308-316 (2011).
7. Reynolds, L. E., et al. Accelerated re-epithelialization in beta3-integrindeficient-mice is associated with enhanced TGF-beta1 signaling. Nat. Med. 11, 167-174 (2005).
8. Munger, J. S., Sheppard, D. Cross talk among TGF-β signaling pathways, integrins, and the extracellular matrix. Cold Spring Harb Perspect Biol. 3, a005017 (2011).
9. Swiecki, M., Colonna, M. Unraveling the functions of plasmacytoid dendritic cells during viral infections, autoimmunity, and tolerance. Immunol. Rev. 234, 142-162 (2010).
10. van Rooij, E., et al. Dysregulation of microRNAs after myocardial infarction reveals a role of miR-29 in cardiac fibrosis. Proc. Natl. Acad. Sci. U.S.A. 105, 13027-13032 (2008).
11. Maurer, B., et al. MicroRNA-29, a key regulator of collagen expression in systemic sclerosis. Arthritis Rheum. 62, 1733-1743 (2010).
12. Hall, J. C., Rosen, A. Type I interferons: crucial participants in disease amplification in autoimmunity. Nat Rev Rheumatol. 6, 40-49 (2010).
13. Sakkas, L. I., Chikanza, I. C., Platsoucas, C. D. Mechanisms of Disease: the role of immune cells in the pathogenesis of systemic sclerosis. Nat Clin Pract Rheumatol. 2, 679-85 (2006).
14. Fujimoto, M., et al. CD19-dependent B lymphocyte signaling thresholds influence skin fibrosis and autoimmunity in the tight-skin mouse. J. Clin. Invest. 109, 1453-1462 (2002).
15. Bona, C, Rothfield, N. Autoantibodies in scleroderma and tight skin mice. Curr Opin Immunol. 6, 931-937 (1994).
16. Brakebusch, C. et al. Skin and hair follicle integrity is crucially dependent on beta 1 integrin expression on keratinocytes. EMBO J. 19(15), 3990-4003 (2000).
17. Liu, S, et al. Loss of beta1 integrin in mouse fibroblasts results in resistance to skin scleroderma in a mouse model. Arthritis Rheum. 60(9), 2817-21 (2009).
18. van Helden, S. F., et al. A critical role for prostaglandin E2 in podosome dissolution and induction of high-speed migration during dendritic cell maturation. J. Immunol. 177, 1567-1574 (2006).
19. Zou, W., et al. Stromal-derived factor-1 in human tumors recruits and alters the function of plasmacytoid precursor dendritic cells. Nat. Med. 7, 1339-1346 (2001).
20. Ding, C., Cai, Y., Marroquin, J., Ildstad, S. T., Yan, J. Plasmacytoid dendritic cells regulate autoreactive B cell activation via soluble factors and in a cell to-cell contact manner. J. Immunol. 183, 7140-7149 (2009).
21. Jego, G., et al. Plasmacytoid dendritic cells induce plasma cell differentiation through type I interferon and interleukin 6. Immunity. 19, 225-234 (2003).
22. Fleming, J. N., et al. Capillary Regeneration in Scleroderma: Stem Cell Therapy Reverses Phenotype. PLoS ONE. 3, e1452 (2008).
23. Saas P, Perruche S. Functions of TGF-β-exposed plasmacytoid dendritic cells. Crit Rev Immunol. 32, 529-53 (2012).
24. Bratke, K., Klein, C., Kuepper, M., Lommatzsch, M., Virchow, J. C. Differential development of plasmacytoid dendritic cells in Th1- and Th2-like cytokine milieus. Allergy. 66, 386-395 (2011).
25. Dardalhon, V., et al. IL-4 inhibits TGF-β-induced Foxp3+ T cells and, together with TGF-β, generates IL-9+ IL-10+ Foxp3− effector T cells. Nature Immunology 9, 1347-1355 (2008).
26. Nakerakanti, S. S., Bujor, A. M., Trojanowska, M. CCN2 is required for the TGF-β induced activation of Smad1-Erk1/2 signaling network. PLoS ONE. 6, e21911 (2011).
27. Chen, Y., et al. Heparan sulfate-dependent ERK activation contributes to the overexpression of fibrotic proteins and enhanced contraction by scleroderma fibroblasts. Arthritis Rheum. 58, 577-585 (2008).
28. Asano, Y., et al. Increased expression of integrin αvβ3 contributes to the establishment of autocrine TGF-β signaling in scleroderma fibroblasts. J. Immunol. 175, 7708-7718 (2005).
29. Watarai, H., et al. PDC-TREM, a plasmacytoid dendritic cell-specific receptor, is responsible for augmented production of type I interferon. Proc. Natl. Acad. Sci. U.S.A. 105, 2993-2998 (2008).
30. Kawai, T., Akira, S. TLR signaling. Cell Death Differ. 13, 816-25 (2006).
31. Judge, D. P. et al. Evidence for a critical contribution of haploinsufficiency in the complex pathogenesis of Marfan syndrome. J. Clin. Invest. 114(2), 172-81 (2004).
32. Castelino, F. V. et al. Amelioration of dermal fibrosis by genetic deletion or pharmacologic antagonism of lysophosphatidic acid receptor 1 in a mouse model of scleroderma. Arthritis Rheum. 63(5), 1405-1415 (2011).
33. HogenEsch, H. et al. Expression of chitinase-like proteins in the skin of chronic proliferative dermatitis (cpdm/cpdm) mice. Exp. Dermatol. 15(10), 808-14 (2006).
34. Davis, E. C. et al. Remodeling of elastic fiber components in scleroderma skin. Connect. Tissue Res. 40(2), 113-21 (1999).
35. Lakos, G. et al. Animal models of scleroderma. Methods Mol. Med. 102, 377-93 (2004).
36. Loeys, B. L., Gerber, E. E., Riegert-Johnson, D. et al. Mutations in fibrillin-1 cause congenital scleroderma: stiff skin syndrome. Sci. Transl. Med. 2, 23ps13 (2010).
37. Garfield, A. S. Derivation of primary mouse embryonic fibroblast (PMEF) cultures. Methods Mol. Biol. 633, 19-27 (2010).
38. Gehrie E et al. Plasmacytoid Dendritic Cells in Tolerance. Methods Mol Biol. 677, 127-47 (2011).
39. Pampori, N. et al. Mechanisms and Consequences of Affinity Modulation of Integrin αvβ3 Detected with a Novel Patch-engineered Monovalent Ligand. J Biol Chem. 274, 21609-21616 (1999).
40. Kiosses, W. B. et al. Rac recruits high-affinity integrin αvβ3 to lamellipodia in endothelial cell migration. Nat Cell. Biol. 3, 316-320 (2001).

While the disclosed method has been described in conjunction with the various exemplary embodiments outlined above, various alternatives, modifications, variations, improvements, and/or substantial equivalents, whether known or presently unforeseen, may become apparent to those having at least ordinary skill in the art. Accordingly, the exemplary embodiments according to this invention, as set forth above, are intended to be illustrative, not limiting.

1. A method of treating a fibrotic disease or condition, comprising administering to a subject having a fibrotic disease or condition an effective amount of an integrin activity-modulating agent, whereby the symptoms of the fibrotic disease or condition are reduced.

2. The method of claim 1, wherein the integrin activity-modulating agent comprises one or more of the following:
a) manganese;
b) an integrin-activating agent or an integrin-blocking agent; and
c) an antibody or a peptide that is capable of interacting with one or more integrins.

3-4. (canceled)

5. The method of claim 2, wherein the antibody or peptide is an integrin-activating antibody or peptide, optionally a β1 integrin-activating antibody or peptide, optionally a β1 integrin-activating antibody (β1aAb), optionally 9EG7 or TS2/16.

6-8. (canceled)

9. The method of claim 2, wherein the antibody or peptide is an integrin-blocking antibody or peptide, optionally a β3 integrin-blocking antibody or peptide, optionally a β3 integrin-blocking antibody (β3bAb).

10-11. (canceled)

12. The method of claim 1, wherein the fibrotic disease or condition is scleroderma.

13. The method of claim 1, wherein the fibrotic disease or condition is selected from the group consisting of stiff skin syndrome, systemic sclerosis, and idiopathic pulmonary fibrosis.

14. An integrin activity-modulating agent for use in treating a fibrotic disease or condition.

15. The integrin activity-modulating agent of claim 14, wherein the integrin activity-modulating agent comprises one or more of the following:
a) manganese;
b) an integrin-activating agent or an integrin-blocking agent; and
c) an antibody or a peptide that is capable of interacting with one or more integrins.

16-17. (canceled)

18. The integrin activity-modulating agent of claim 15, wherein the antibody or peptide is an integrin-activating antibody or peptide, optionally a β1 integrin-activating antibody or peptide, optionally a β1 integrin-activating antibody (β1aAb), optionally 9EG7 or TS2/16.

19-21. (canceled)

22. The integrin activity-modulating agent of claim 15, wherein the antibody or peptide is an integrin-blocking antibody or peptide, optionally a β3 integrin-blocking antibody or peptide, optionally a β3 integrin-blocking antibody (β3bAb).

23-24. (canceled)

25. The integrin activity-modulating agent of claim 14, wherein the fibrotic disease or condition is scleroderma.

26. The integrin activity-modulating agent of claim 14, wherein the fibrotic disease or condition is selected from the group consisting of stiff skin syndrome, systemic sclerosis, and idiopathic pulmonary fibrosis.

27. An integrin activity-modulating agent for use in manufacturing a medicament for treating a fibrotic disease or condition.

28. The integrin activity-modulating agent of claim 27, wherein the integrin activity-modulating agent comprises one or more of the following:
a) manganese;
b) an integrin-activating agent or an integrin-blocking agent; and
c) an antibody or a peptide that is capable of interacting with one or more integrins.

29-30. (canceled)

31. The integrin activity-modulating agent of claim 28, wherein the antibody or peptide is an integrin-activating antibody or peptide, optionally a β1 integrin-activating antibody or peptide, optionally a β1 integrin-activating antibody (β1aAb), optionally 9EG7 or TS2/16.

32-34. (canceled)

35. The integrin activity-modulating agent of claim 28, wherein the antibody or peptide is an integrin-blocking antibody or peptide, optionally a β3 integrin-blocking antibody or peptide, optionally a β3 integrin-blocking antibody (β3bAb).

36-37. (canceled)

38. The integrin activity-modulating agent of claim 27, wherein the fibrotic disease or condition is scleroderma.

39. The integrin activity-modulating agent of claim 27, wherein the fibrotic disease or condition is selected from the group consisting of stiff skin syndrome, systemic sclerosis, and idiopathic pulmonary fibrosis.


SEQUENCE LISTING (2 sequences)

SEQ ID NO: 1 - DNA, 18 bp, Artificial Sequence
  Forward PCR primer
gatcccaccacctgcatc

SEQ ID NO: 2 - DNA, 21 bp, Artificial Sequence
  Reverse PCR primer
catgtgttcacagaaggacac